(19) United States
(12) Patent Application Publication
(10) Pub. No.: US 2016/0025744 A1
(43) Pub. Date: Jan. 28, 2016

(54) PLASMONIC SUBSTRATE FOR MULTIPLEX ASSESSMENT OF TYPE 1 DIABETES

(71) Applicant: THE BOARD OF TRUSTEES OF THE LELAND STANFORD JUNIOR UNIVERSITY, Palo Alto, CA (US)

(72) Inventors: Brian Jay FELDMAN, Portola Valley, CA (US); Hongjie DAI, Cupertino, CA (US); Rajiv B. KUMAR, Palo Alto, CA (US); Bo ZHANG, Stanford, CA (US)

(21) Appl. No.: 14/774,658

(22) PCT Filed: Mar. 13, 2014
(86) PCT No.: PCT/US14/25977

§ 371 Date: Sep. 10, 2015

Related U.S. Application Data
(60) Provisional application No. 61/783,605, filed on Mar. 14, 2013.
(60) Provisional application No. 61/911,315, filed on Dec. 3, 2013.

(51) Int. Cl. G01N 33/68

(52) U.S. Cl. CPC G01N 33/6854; G01N 2800/042

(57) ABSTRACT

Disclosed are methods and materials providing fluorescence detection of autoantibodies present in individuals who have developed or are at risk for type 1 diabetes. Provided is a plasmonic chip capable of fluorescence-enhancement of >100-fold. The fluorescent signal is generated by an anti-human antibody antibody, such as an anti-IgG antibody that is coupled to a fluorophore selected to emit at a wavelength enhanced by the plasmonic chip, for example in the NIR.

### CROSS-REFERENCE TO RELATED APPLICATIONS

This application claims priority from U.S. Provisional Patent Application No. 61/783,605, filed Mar. 14, 2013, and U.S. Provisional Patent Application No. 61/911,315, filed Dec. 3, 2013, which are hereby incorporated by reference in their entirety.

### STATEMENT OF GOVERNMENTAL SUPPORT

This invention was made with Government support under contract TW008781 and contract CA135109 awarded by the National Institutes of Health. The Government has certain rights in this invention.

### REFERENCE TO SEQUENCE LISTING, COMPUTER PROGRAM, OR COMPACT DISK

None.

### BACKGROUND OF THE INVENTION

1. Field of the Invention

The present invention relates to the formation of nanostructured plasmonic metal films on substrates, where such plasmonic films are useful for spectroscopy and immunoassays, and, in particular to a plasmonic substrate used for detection of autoantibodies indicative of type I diabetes.

2. Related Art

Presented below is background information on certain aspects of the present invention as they may relate to technical features referred to in the detailed description, but not necessarily described in detail. That is, individual compositions or methods used in the present invention may be described in greater detail in the publications and patents discussed below, which may provide further guidance to those skilled in the art for making or using certain aspects of the present invention as claimed. The discussion below should not be construed as an admission as to the relevance or the prior art effect of the patents or publications described.

Diabetes is the second most common chronic disease of children, and the incidences of both type 1 and type 2 diabetes are rising worldwide. The prevalence of autoimmune type 1 diabetes (T1D) in children is predicted to increase by 70% between 2004 and 2020, with twenty-five percent of affected children in South-east Asia. The incidence of type 2 diabetes (T2D) in children, along with the prevalence of obesity, has also been increasing exponentially since the early 1990s. The SEARCH for Diabetes in Youth epidemiologic study in the US demonstrated that about 25% of new diabetics less than 20 years in age have type 2. This concurrent rise has resulted in a worldwide diagnostic dilemma and it is no longer clear what type of diabetes a symptomatic child has at disease presentation.

Children with new-onset T1D require multi-daily insulin injection therapy to prevent diabetic ketoacidosis (DKA)—a life-threatening condition. Children with T2D can generally be treated as out-patients with diet, exercise and oral medication. Traditionally in the US, all children symptomatic with new-onset diabetes (polyuria, polydipsia and hyperglycemia) are admitted to the hospital for initiation of insulin therapy to prevent potential decline to DKA should they have T1D. As a result, up to 25% of children have unnecessary stress and health care expenditure. In comparison, a symptomatic child in the developing world, where T2D is more prevalent than T1D, may not have access to insulin until symptoms have progressed to DKA from unrecognized T1D. Given that up to 98% of T1D patients are positive for an autoantibody to one or more pancreatic islet antigen (insulin, GAD65 and/or IA2), all symptomatic children in the US are tested by radioimmunoassay (RIA) for these biomarkers to differentiate T1D and T2D. Unfortunately, this testing is cumbersome, is not available in resource-poor settings, and results are not available until weeks after a treatment plan has been selected.

Although RIA provides sensitive detection of diabetes autoantibodies, it requires radioisotopes, is time intensive, is expensive ($300-400), does not allow antigen multiplexing, and requires venipuncture. Additionally, the Diabetes Autoantibody Standardization Program (DASP) workshops have reported unfavorable reviews of non-central laboratory RIA kits.

The current standard for differentiating type 1 (autoimmune) from type 2 (and other nonautoimmune forms of diabetes) is to test for the presence of one or more diabetes autoantibody (IA2 Ab, ICA512 Ab, GAD65 Ab, and ZnT8 Ab). The detection of a single diabetes autoantibody is diagnostic for type 1 diabetes. The current standard is performed with a radioimmunoassay, is very costly, cannot be multiplexed, is very expensive, and is not possible to perform in primary care and ER settings where it would be most effective and clinically useful. More traditional and cheaper methods for detecting autoantibodies, including ELISA, have been unsuccessful.

### SPECIFIC PATENTS AND PUBLICATIONS

Tabakman S M, Lau L, Robinson J T, et al. “Plasmonic substrates for multiplexed protein microarrays with femtomolar sensitivity and broad dynamic range,” Nature Communications 13 Sep. 2011; 2(466) discloses protein microarrays on a novel nanostructured, plasmonic gold film with near-infrared fluorescence enhancement of up to 100-fold. Plasmonic protein microarrays for the early detection of a cancer biomarker, carcinoembryonic antigen, in the sera of mice bearing a xenograft tumour model were demonstrated.

U.S. patent application Ser. No. 13/728,798, filed Dec. 27, 2012, claiming benefit to U.S. provisional patent application 61/580,883, filed Dec. 28, 2011, refers to the above-referenced publication and is hereby specifically incorporated by reference.

Hong, G. S. et al. “Near-Infrared-Fluorescence-Enhanced Molecular Imaging of Live Cells on Gold Substrates,” Angew Chem Int Edit 50, 4644-4648 (Apr. 19, 2011) contains a description by the present inventors of the present Au/Au films prepared on quartz through solution phase growth, and or SWBT-IR800-RGD conjugates.

Tabakman, S. M., Chen, Z., Casalongue, H. S., Wang, H. L. & Dai, H. J. A New Approach to Solution-Phase Gold Seeding for SERS Substrates. Small 7, 499-505 (3 Jan. 2011) contains a description by the present inventors of the presently used solution phase gold seeding to create a highly stable SERS-active gold substrate.

### BRIEF SUMMARY OF THE INVENTION

The following brief summary is not intended to include all features and aspects of the present invention, nor does it imply that the invention must include all features and aspects discussed in this summary.

The present invention comprises, in certain aspects, a biosensor for use in a spectroscopic detection system, comprising: (a) a substrate; (b) a discontinuous gold film applied to said substrate, said gold film having plasmonic nano-islands of gold grown on gold seeds; and (c) an array of antigens disposed in discrete locations and coupled to the discontinuous gold film, whereby emission from a label bound to an analyte capture agent is enhanced by the discontinuous gold film, wherein said antigens are at least two of (i) GAD65 (glutamic acid decarboxylase-65 kDa); (ii) GAD67 (glutamic acid decarboxylase-67 kDa); (iii) IA512 (islet cell autoantigen 512); (iv) IA-2 (insulinoma antigen 2); (v) ZnT8 (zinc transporter 8); and (vi) human insulin or an immunologically active fragment thereof.

In certain aspects, the biosensor of the present invention is also one wherein said antigens comprise purified recombinant proteins. The present invention comprises, in certain aspects a biosensor wherein the purified recombinant antigens are at least three of, at least four of, at least five of, or at least six or, the antigens selected from antigens (i) through (vi). In certain aspects, the biosensor of the present invention is also one wherein the antigens are chemically linked to the plasmonic nanoislands by a branched polyethylene glycol. In certain aspects of the invention the antigens further include an antigen that is reactive to antibodies raised by a vaccine. In certain aspects of the invention the antigen, which is used as a positive control, is tetanus toxoid, since the subjects will have received tetanus vaccine and therefore will have antibodies to tetanus toxoid.

In certain aspects of the invention, the biosensor further comprises channels on the biosensor for delivering reagents to said array of antigens. In certain aspects of the invention the biosensor further comprises a separation zone for separating blood components and passing serum containing autoantibodies through the channels, whereby whole blood can be introduced into the biosensor. In certain aspects of the invention the biosensor further comprises channels, in use, that contain an anti-human IgG composition.

The present invention comprises, in certain aspects a method for determining an antibody specificity profile in a patient having a predisposition to insulin-dependent diabetes mellitus (IDDM), comprising: (a) providing an array comprising at least three purified proteins selected from the group consisting of: (i) GAD65 (glutamic acid decarboxylase-65 kDa); (ii) GAD67 (glutamic acid decarboxylase-67 kDa); (iii) IA512 (islet cell autoantigen 512); iv) IA-2 (insulinoma antigen 2); (v) ZnT8 (zinc transporter 8); and (vi) human insulin or an immunologically active fragment thereof; (b) said array coupled to a plasmonically active gold film in individual spots of arrayed proteins; (c) contacting the array from step (a) with a patient sample comprising antibodies; (d) identifying antigens in the array that bind to antibodies within the patient sample contacted in step (c) with a labeled antibody that binds to human antibodies and carries a fluorescent dye whose fluorescence is enhanced by the plasmonically active gold film. In certain aspects of the invention the above methods further comprise reading fluorescence in a fluorescent reader that directly exposes the array to NIR and receives NIR reflected from individual spots. In certain aspects of the invention the above method further comprises the step of quantifying levels of antibody to said at least three antigens. In certain aspects of the invention the above method further comprises use of least three purified proteins are recombinant proteins.

In certain aspects of the invention the above method further comprises: providing a biosensor for use in a spectroscopic detection system, comprising: (i) a substrate; (ii) a discontinuous gold film applied to said substrate, said gold film having plasmonic nano-islands of gold grown on gold seeds; and (iii) an array of antigens disposed in discrete locations and coupled to the discontinuous gold film, whereby emission from a label bound to an analyte capture agent is enhanced by the discontinuous gold film, wherein said antigens are at least two of (i) GAD65 (glutamic acid decarboxylase-65 kDa); (ii) GAD67 (glutamic acid decarboxylase-67 kDa); (iii) IA512 (islet cell autoantigen 512); (iv) IA-2 (insulinoma antigen 2); (v) ZnT8 (zinc transporter 8); and (vi) human insulin protein or an immunologically active fragment of said insulin protein; and (vii) an antigen reactive to antibodies commonly found in humans; contacting said array of antigens with a subject sample containing antibodies; preparing complexes of antigens binding to antibodies in step (b); labeling complexes from step (c) with a florescent label enhanced by the plasmonically active gold film; and (e) measuring fluorescence level of said complex as labeled in step (d) and comparing florescence levels between at least two antigens selected from (i) through (vii).

The present invention incorporates protein microarray printing on a gold plasmonic substrate and allows for multiplexed testing for more than one diabetes autoantibody from a single patient sample, in addition to testing for diagnostic autoantibodies for other autoimmune diseases known to have increased prevalence in patients with type 1 diabetes from the same sample (including celiac disease, autoimmune hypothyroidism, and Addison disease). The invention further comprises stratifying detection of IgG autoantibodies from IgA autoantibodies by use of fluorescent detection labels of different frequencies to allow celiac disease testing on the same blood sample and same slide for a given patient.

The present invention further comprises a microfluidic chip containing diabetes antigens and control antigens, and channels for flowing solutions and anti-human IgG solutions onto these antigens. The chip is configured to be read directly in a NIR fluorescence reader that will detect the various antigens. The chip containing the arrayed antigens is designed so that the spots on the array may be directly exposed to NIR and the fluorescent signal from each spot read. This may be done in a variety of ways, for example, by forming the chip with a window above the spots, where the window is transparent to NIR, such as high density polyethylene. This, coupled with a simple chip reader, provides a point of care system. Further details on point of care diagnostics may be found, e.g. at Chin C D, Linder V, Sia S K. Lab-on-a-chip devices for global health: past studies and future opportunities. Lab Chip 7, 41-57(2007). The chip reader will include a fluorescence measurement device reading in the NIR range.

The following examples illustrate novel applications of plasmonic gold substrates for near-infrared fluorescence enhanced detection [see Tabakman, S. M., et al. Plasmonic substrates for multiplexed protein microarrays with femtomolar sensitivity and broad dynamic range. Nat. Commun. 2, 466 (2011); Zhang, B., et al. Multiplexed cytokine detection on plasmonic gold substrates with enhanced near-infrared fluorescence. Nano Research 6, 113-120 (2013).].

The present diabetes autoantibody platform overcomes the critical challenges to rapid, sensitive and specific diagnosis of diabetes. This approach allows successful detection of T1D autoantibodies in ultralow volumes of human serum, such as that obtained from a finger-prick sample, with results available after a short processing time. The platform has been tested on 26 insulin-naïve children with new-onset T1D, and 13 children with new-onset T2D. The tests found at least equivalent sensitivity and specificity when compared with RIA. Additionally, the present assay permits multiplexing of the islet antigens, provides autoantibody signal quantification, detects several isotypes of autoantibodies via multi-colors on a single chip, is inexpensive and, importantly, provides a point-of-care platform. It is known that traditional protein assays that use optical reporters, including ELISA and protein microarrays on nitrocellulose or glass substrates, fail to reliably detect diabetes autoantibodies. The low signal and high background noise when these assays are attempted to measure islet cell autoantibodies is a likely cause of these failures. This is further confounded by the inherent challenge of maintaining the native structure of the insulin epitope binding sites when immobilizing the antigen on a solid surface. The unique surface chemistry of the present plasmonic gold chip platform permits immobilization of islet antigens while preserving antibody binding. In addition, this surface chemistry maximizes analyte capture while resisting non-specific binding resulting in low background and a diagnostic sensitivity equivalent to RIA. Further, the plasmonic gold chips have a batch-to-batch CV<10% at low analyte concentrations (down to 0.1 U/mL) (FIGS. 7C, D and FIG. 15). The NIR-fluorescence enhancement (NIR-FE) on plasmonic gold films emanates from local electric field enhancement by the abundant nanoscale-gaps between gold nanoislands, and enhanced radiative decay rates or fluorescence quantum yield due to resonance emission dipole coupling to plasmonic modes. The NIR-FE of positive signals, combined with low background non-specific signals are responsible for the broad dynamic range and lower limit of detection compared to conventional substrates, leading to an improved sensitivity in T1D diagnosis (FIG. 7 and Table 1). This is the first time that protein microarrays on plasmonic gold chips are used for human disease diagnosis.

Further, the ability to perform isotype specific analysis enabled our identification of IgM and IgA isotype autoantibodies. We are not aware of any previous report of multiplexed detection of these isotypes in a single assay platform for a diabetic patient, demonstrating how this platform can be used as a new research tool to test for putative novel biomarkers. The identification of biomarkers that can detect acute islet cell injury earlier than those currently used would significantly advance the field by facilitating an array of basic and clinical research opportunities.

The plasmonic gold platform allows simultaneous detection and quantification of autoantibodies to islet antigens and their isotypes. It requires only a 2 μL sample of whole blood without the need of further processing to obtain serum. This can be obtained from a simple finger-prick, which opens the possibility of point-of-care T1D diagnosis.

In addition to addressing the current clinical need for improved diabetes diagnostics, we present technology can enable a broad range of advances in basic and clinical research that were not previously feasible. For example, serial monitoring of autoantibodies in patients undergoing novel interventions might predict efficacy, as therapies that protect against continued islet cell destruction may correlate with a decline in autoantibody titer. In addition, following antibody levels at high resolution in patients considered ‘high-risk’ by genetics would yield great insight into the natural history of the development of diabetes. Ultimately, we believe this technology could be deployed to facilitate screening for islet antigen autoantibodies, identifying those who would otherwise be at-risk for progression to DKA, and allowing the testing of preventative interventions prior to the onset of clinical symptoms.

### BRIEF DESCRIPTION OF THE DRAWINGS

FIG. 1 is a schematic illustration of a nanostructured plasmonic gold film prepared as a platform for protein detection with 100-1000 fold enhanced NIR fluorescence and sensitivity. It shows a plasmonic gold substrate upon which has been coated a layer of PEG star, i.e. polyethylene glycol having 10 to 100 PEG chains emanating from a central core group. The antigen spots are deposited on the PEG star layer.

FIG. 2 shows results from a prior assay for CEA, by way of illustration; shown are images of multiplexed autoantigen arrays probed with human serum containing autoantibodies on a gold substrate. (Tabakman, et. el., Nature Comm, 2, 466 (2011)).

FIG. 3A-D. FIG. 3A: Schematic drawing of the layout for a diabetes microarray; antigens are printed in triplicate. Diabetes antigens are insulin, GAD and IA-2. FIG. 3B: Digital image showing the low volume of sample (2 uL) needed to profile autoantibodies in human blood or serum. FIG. 3C: Typical IR800 fluorescent image reflecting autoantibodies profiling result in T1D patient (left, with GAD positive) and non-T1D patient (right). FIG. 3D: A schematic drawing of a microfluidic chip for use with the present assay, showing fluid flow zones.

FIG. 4A, 4B: Diabetes microarray on plasmonic substrate can be applied for future point-of-care devices. FIG. 4A: Typical fluorescence image for profiling autoantibodies in blood (top) and serum (bottom) of the same T1D patient. FIG. 4B: bar chart for quantifying mean fluorescence for antibody reactivity against the 3 antigens in blood and serum.

FIG. 5A, 5B. FIG. 5A: fluorescence images for probing IA-2 array with serial dilution of human anti IA-2 antibody. FIG. 5B: calibration curve from FIG. 5A, demonstrating that plasmonic substrate provides a more sensitive autoantibody measurement with sensitivity below 0.01 U/ml (Cut off for RIA is 1 U/ml).

FIGS. 6A-1, 6A-2, 6B-1, 6B-2, 6C-1, and 6C-2 show results from different patient samples quantifying diabetes autoantibodies by plasmonic substrate and by RIA. Results were obtained from different patient samples quantifying human IgG autoantibodies against insulin by plasmonic substrate (FIG. 6A-1) and by RIA (FIG. 6A-2). The horizontal line near the bottom of the graph line represents the cut off for plasmonic Au through mean value of the T2D (type 2 diabetes) signal plus 2 standard deviations. Results were also obtained for the same patients detecting antibodies to GAD (FIG. 6B-1, 6B-2) and IA-2 (FIG. 6C-1, 6C-2), where improved sensitivity was shown by the plasmonic Au substrate.

FIGS. 7A, 7B, 7C-1, 7C-2, 7C-3, and 7D: Greater signal detection on plasmonic gold surface in comparison to standard surfaces. FIG. 7A: Electron micrograph demonstrating gold islands and abundant nanogaps in the nanostructured gold plasmonic film. FIG. 7B: Schematic depicting the spatial relationship of the platform's PEG layer, the islet-specific antigens, the primary autoantibodies (Ab) from diluted human serum or blood, and the detection antibodies conjugated with a fluorophore signal. FIG. 7C: Calibration curves showing comparison of detection limit and dynamic range of islet antigen autoantibody quantification on plasmonic gold, glass and evaporated gold substrates (FIG. 7C-1 shows insulin autoantibody, FIG. 7C-2 shows GAD65 autoantibody, and FIG. 7C-3 shows IA2 autoantibody). Samples used for the calibration curves were standards containing known concentrations of autoantibodies in serum provided by the vendor (Kronus Inc). Concentrations of standard samples for auto-insulin were in vendor's units (KU/mL), and concentrations of standard samples for autoantibodies against GAD64 and IA-2 are in international units of U/mL. Three independent experiments were performed. Data shown as the mean+−s.d. D) Comparison of detection limits and dynamic ranges of insulin islet autoantibodies quantification between plasmonic gold and RIA. RIA data was from vendor provided RIA kits. Data shown as the mean+−s.d.

FIG. 8A-8D: The plasmonic chip readily differentiates T1D and T2D in ultra low serum or blood samples. Fluorescence mapping result (FIG. 8A) and signal quantification (FIG. 8B) on plasmonic gold chips for islet antigen autoantibody detection of typical T1D and T2D patients or non-diabetic controls (control). Comparison of signal on chips tested with whole blood or serum from a typical subject (FIG. 8C) and quantification of signals comparing whole blood and serum in four independent patients (FIG. 8D). Three independent experiments were performed. Data shown as the mean+−s.d.

FIG. 9: Scatter plot for diabetes autoantibodies. Analysis of our subject pool of 26 children with new-onset T1D (squares), 13 children with new-onset T2D (circles) and 5 non-diabetic children (diamonds) demonstrates a specific (non-normal) distribution of MFI values for the T1D patients. Each point represents an individual patient. The titer for each of the 3 autoantibodies for the individual patient is plotted on the 3-dimensional axes. Points that fall within the blue box are negative for T1D by plasmonic gold platform testing.

FIG. 10A-10D: The plasmonic chip permits differentiation of immunoglobulin isotypes from a single ultra low volume sample. 10A) Absorbance and fluorescence emission of fluorophore Cy3, Cy5 and IRDye800. The emission spectrum of the 3 fluorophores are non-overlapping with the emission spectrum at the trough of the absorbance spectrum (black), preventing absorption by fluorophores with adjacent emission spectrum. 10B) Simultaneous specific detection of IgG, IgM and IgA isotypes on a multiplexed plasmonic gold chip using secondary antibodies with narrow non-overlapping emission spectra. 10C) (left) Key for multiplexed chips. (right) Multiplexed detection of islet cell autoantibody isotypes in a child with new-onset T1D. 10D) The multiplexed plasmonic gold chip simultaneously detects specific IgG autoantibodies to GAD65 and IA2 and IgM autoantibodies against insulin and GAD65 from a single sample in the example patient.

FIG. 11A, 11B: Optimization of human serum incubation condition. Fluorescence images (11A) and background signal plot (11B) demonstrate that, compared to whole serum incubation or 1:1 dilution in FBS, 1:10 dilution of human serum in whole FBS improves signal/background ratio of the microarray by vastly decreasing background caused by non-specific binding (NSB).

FIG. 12A, 12B: Islet antigen microarray performance on plasmonic chip, nitrocellulose substrate and glass substrate. Fluorescence mapping results (FIG. 12A) and signal/background ratios (FIG. 12B) obtained with an example patient's serum sample on the three different substrates, showing that insulin autoantibodies were only detectable on the plasmonic platform. Also, the NIR fluorescence signal-to-noise ratios for the detection of GAD65 and IA2 autoantibodies were far superior on the gold plasmonic platform compared to glass and nitrocellulose.

FIG. 13: Fluorescence enhancement of Cy3, Cy5 and IRDye800 on plasmonic gold substrates. Mean fluorescence intensity of Cy3/Cy5/IRDye800 for monolayer of avidin conjugated Cy3/Cy5/IRDye800 on gold plasmonic (Au) compared to glass substrate. Fluorescence was measured with GenePix4000B for Cy3 and Cy5 fluorescence and a Li-Cor Odyssey scanner for the IRDye800 fluorescence. A ˜3, ˜50 and ˜100 fold enhancement was observed for Cy3, Cy5 and IRDye800 respectively on the plasmonic gold compared to glass substrate.

FIG. 14: Immobilization of insulin on gold plasmonic substrates treated with different surface chemistry for autoantibody detection. Top panel: Cy5 fluorescence images and bottom panel: IR800 signal quantification for detecting insulin autoantibodies in the serum of a T1D patient. Cy5-labeled insulin was spotted onto plasmonic gold substrate (through microarray printing, see online method) with different surface modifications as indicated (bare, branched-PEG coating and aluminum oxide coating respectively) followed by incubation with serum from T1D patient known to have insulin autoantibody and subsequent detection with IRDye800 labeled anti human IgG secondary antibody. The fluorescence intensity of Cy5 shown in the bar graph reflects the amount of insulin immobilized on the plasmonic substrate while the fluorescence intensity of IRDye800 reflects the amount of insulin autoantibody captured on the insulin spot. While more insulin was immobilized on bare Au compared to on Au-Branched PEG, fewer autoantibodies were detected. This suggests that insulin proteins on bare gold are less well recognized by autoantibodies than insulin on a PEG cushion layer on gold because of changes in the protein tertiary structure. No insulin is immobilized on the Au −5 nm Al₂O₃ surface after incubation in human serum and secondary antibody solution, suggesting weak binding of insulin to aluminum oxide resulting in loss of protein by solution rinsing.

FIG. 15A-15C: Reproducibility of the plasmonic gold chip platform for detecting islet autoantibodies. Islet autoantibodies were measured in every diabetic patient in 3 independent experiments (dotted/thin/thick bars for each patient) using 3 different chips made by the identical reaction steps and conditions. The quantified IRDye800 signal for each islet antigen was highly reproducible across the independent experiments. FIG. 15A shows insulin Ab; FIG. 15B shows Gad65 Ab, and FIG. 15C shows IA2 Ab, in different patients

FIGS. 16A-16C: Quantification of plasmonic gold signal for 3 autoantibodies for every patient. Diabetes autoantibody titers were measured and compared in 26 patients with T1D (left side set bars), 13 patients with T2D (middle set bars), and 5 non-diabetic volunteers (right set bars). FIG. 16A shows insulin Ab; FIG. 16B shows Gad65 Ab, and FIG. 16C shows IA2 Ab, in different patients.

### DETAILED DESCRIPTION OF THE PREFERRED EMBODIMENT

### Definitions

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well-known and commonly used in the art. Certain experimental techniques, not specifically defined, are generally performed according to conventional methods well known in the art and as described in various general and more specific references that are cited and discussed throughout the present specification. For purposes of clarity, the following terms are defined below.

Ranges: For conciseness, any range set forth is intended to include any sub-range within the stated range, unless otherwise stated. As a non-limiting example, a range of 120 to 250 is intended to include a range of 120-121, 120-130, 200-225, 121-250 etc. The term “about” has its ordinary meaning of approximately and may be determined in context by experimental variability. In case of doubt, “about” means plus or minus 5% of a stated numerical value.

The term “antibody” means any of several classes of structurally related proteins, also known as immunoglobulins, that function as part of the immune response of an animal, which proteins include IgG, IgD, IgE, IgA, IgM (“isotypes”) and related proteins which specifically bind to their cognate antigens. The term “antibody” here refers to an antibody specifically binding to a single antigen specificity rather than a mixed population of antibody specifies. Antibodies as contemplated herein are any antibody-like molecule useful in an immunoassay, including known direct and indirect (“sandwich”) immunoassays. The term antibody as used herein refers to naturally arising human antibodies, as well as synthetic mimics which may be used for detection antibodies. As will be discussed below, IgG, IgA, IgM etc. are different molecules.

Both IgM and IgG refer to a class of immunoglobulin. IgM refers to those antibodies that are produced immediately after an exposure to the disease, while IgG refers to a later response. IgG generally confers immunity to a patient so far as that particular disease is concerned. IgG may also be discriminated into subclasses IgG1 through 4.

The term “specific binding” means that binding which occurs between such paired species as enzyme/substrate, receptor/agonist or antagonist, antibody/antigen, complementary polynucleotides (polynucleic acids) and lectin/carbohydrate which may be mediated by covalent or non-covalent interactions or a combination of covalent and non-covalent interactions. When the interaction of the two species produces a non-covalently bound complex, the binding that occurs is typically electrostatic, hydrogen-bonding, or the result of lipophilic interactions. Accordingly, “specific binding” occurs between a paired species where there is interaction between the two, which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

The term “plasmonically active” in reference to a material means a material which supports plasmons, particularly surface plasmons, thereby exhibiting plasmonic properties. Surface plasmons have been used to enhance the surface sensitivity of several spectroscopic measurements (observing a spectrum of light) including fluorescence, Raman scattering, and second harmonic generation. The term may be more fully understood by reference to Wilson et al. “Directly fabricated nanoparticles for Raman scattering,” US Pub. 20110250464.

The phrase “plasmonic properties” refers to properties exhibited by surface plasmons, or the collective oscillations of electrical charge on the surfaces of metals. In this sense, plasmonic properties refers to measurable properties, as described e.g. in Nagao et al. “Plasmons in nanoscale and atomic-scale systems,” Sci. Technol. Adv. Mater. 11 (2010) 054506 (12pp), describing plasmonic sensors, such as those used for surface-enhanced IR absorption spectroscopy (SEIRA), surface-enhanced Raman scattering (SERS). Another plasmonic property is plasmon-enhanced fluorescence, described e.g. in Sensors and Actuators B 107 (2005) 148-153. That study presented a combination of a nanosphere lithography technique and a surface-enhanced fluorescence technique as a strategy to increase the sensitivity of biochips based on the fluorescent dye Cy5.

The term “metal enhanced fluorescence” or “MEF” is used in its commonly accepted sense of an enhancement of fluorescent intensity of a fluorophore in proximity to a metal where fluorophores in the excited state undergo near-field interactions with the metal particles to create plasmons. The enhancement results from plasmon-coupling and amplification.

The term “NIR label” means a near infrared label, such as carbocyanine dye (for example, an indocyanine dye), that optically comprises a functional group, for example, a succinimidyl ester, that facilitates covalent linkage to a cellular component. Exemplary dyes include, for example, Cy5, Cy5.5, and Cy7, each of which are available from GE Healthcare; VivoTag-680, VivoTag-5680, VivoTag-5750, each of which are available from VisEn Medical; AlexaFluor660, AlexaFluor680, AlexaFluor700, AlexaFluor750, and Alexa Fluor790, each of which are available from Invitrogen; Dy677, Dy676, Dy682, Dy752, Dy780, each of which are available from Dyonics; DyLight547 and DyLight647, each of which are available from Pierce; HiLyte Fluor 647, HiLyte Fluor 680, and HiLyte Fluor 750, each of which are available from AnaSpec; IRDye800CW, IRDye 800RS, and IRDye 700DX, each of which are available from Li-Cor; and ADS780WS, ADS830WS, and ADS832WS, each of which are available from American Dye Source. NIR labels can be enhanced by metal-enhanced fluorescence (MEF), whereby metallic nanostructures favorably modify the spectral properties of fluorophores and alleviate some of their more classical photophysical constraints. This is described further in Geddes, C. D.; Lakowicz, J. R. Metal-enhanced fluorescence. J. Fluoresc. 2002, 12, 121-129. As opposed to other NIR active materials such as carbon nanotubes, the present dyes are water soluble. The preferred dye, IR800 fluoresces at about 800 nm.

The term “NIR” means near infra-red, particularly in the sense of NIR fluorescence. The term also means the near infra-red region of the electromagnetic spectrum (from 0.75 to 3 μm). For purposes of biological imaging, the NIR range is divided into NIR-I, around 800 nm (0.75-0.9 μm) and NIR-II, between about 1 (e.g. 1.1) and 1.4 μm.

MEF (metal enhanced fluorescence) is achieved here by design of the gold islands with sizes and gaps that enhance the local excitation of electric fields, thus enhancing excitation. The plasmonic modes in the gold islands are also tuned to couple resonantly to the emission dipoles of the fluorophores, leading to enhanced radiative decay and thus increased fluorescent quantum yield. Fluorescence enhancement by ˜100-fold can be achieved. The plasmonic resonance wavelengths of the gold film were tunable by the precipitation and seeding parameters and can span from ˜500 nm to ˜2 μm, which overlaps with the excitation and emission energies of many fluorophores. Due to the enhanced excitation electric fields by nanogaps and resonant fluorophore emission coupling to the plasmonic modes, the fluorescence of several NIR agents was experimentally enhanced by ˜15- to 100-fold for several fluorophores, including Cy5 and IRdye-800. The present gold-on-gold methods produce random gold nano-islands at ˜10-100 nm nano-gap spacing, and plasmonic peaks in the 525-1400 nm range useful for fluorescence enhancement. Au films are known to exhibit plasmon resonances at longer wavelengths than silver (Ag) due to higher dielectric constants. Gold films will afford at least MEF of at least 2 fluorophores or 2 colors in the 700-900 nm emission range. For the short wavelength emitting dyes such as Cy3, films with mixed Ag and Au nanostructures on glass may be prepared, in self-assembled arrangement or by patterning techniques. It is possible to pattern Ag and Au in regular arrays at designed locations for making Ag/Au plasmonic films on glass for fluorescence enhancement in a wide spectral range of 500-900 nm, or even from the 500-1400 nm infrared range.

The term “self-assembled monolayer” (SAM) refers to a spontaneously adsorbed monolayer film as is known to assemble onto a gold surface. This has been demonstrated for a wide variety of functional groups such as sulfides, phosphines, thiols, and disulfides. Particularly included are SAMs that reveal a SERS signal, such as 4-mercaptobenzoic acid (4-MBA) self-assembled monolayers on gold substrates. Also preferred is a benzenethiol SAM. Benzenethiol is also a Raman-active molecule capable of forming a SAM. Like 4-MBA, it's useful for probing the extent of SERS from the gold film in a controllable way. Benzene thiol SAMs are further described e.g. in U.S. Pat. No. 6,755,953, entitled “Method for forming ordered structure of fine metal particles,” issued Jun. 29, 2004.

The terms “HLA-DR3-DR4” refers, as understood in the art, to specific HLA types HLA-DR3 and HLA-DR4. Among people with type 1 diabetes, 95% have HLA-DR3, HLA-DR4, and a specific HLA-DQ-Beta. HLA-DR3-DR4 may be determined and their status with the antibody status determined as described below.

### General Method and Materials

The multiplexed protein microarray on a plasmonic gold substrate here is applied to rapid identification of diabetes autoantibodies. The current standard for differentiating type 1 (autoimmune) from type 2 (and other non-autoimmune forms of diabetes) is to test for the presence of one or more diabetes autoantibodies. Exemplary antibodies are antibodies to IA2 (insuloma antigen 2), described, e.g. in Batstra et al., “Low prevalence of GAD and IA2 antibodies in schoolchildren from a village in the southwestern section of the Netherlands,” Hum Immunol. 2001 October; 62(10):1106-10, antibodies to ICA512 (islet cell autoantigen 512, described e.g. in Solimena et al., “ICA 512, an autoantigen of type I diabetes, is an intrinsic membrane protein of neurosecretory granules, EMBO J. 1996 May 1; 15(9): 2102-2114.), antibodies to GAD65 (glutamic acid decarboxylase-65, UNIPROT entry Q99259 or Q05329), antibodies to ZnT8 (zinc transporter 8, described e.g. in Enee et al., “ZnT8 is a major CD8+ T cell-recognized autoantigen in pediatric type 1 diabetes,” Diabetes. 2012 July; 61(7):1779-84. Epub 2012 May 14.

EP 2302387, “Therapeutic and diagnostic uses of antibody specificity profiles for insulin-dependent diabetes mellitus,” describes certain methodologies for determining an antibody profile in a subject to be tested with the present highly sensitive biosensors. Described therein is a method for determining the antibody specificity profile in an individual. This specificity profile reveals the individual's immune response to multiple antigens and/or epitopes of autoantigens, allergens, graft antigens, etc. The antibody specificity profile is determined through the binding of patient samples comprising antibodies to the arrays. The array can comprise antigens and epitopes. Antigen panels or arrays for insulin dependent diabetes mellitus may comprise the antigens and epitopes derived from IA-2; IA-2beta; GAD; insulin; proinsulin; HSP; glima 38; ICA69; and p52. Purified ICA 512 protein may be prepared as described in Solimena et al. supra.

Subjects can be tested using the present biosensor at a pre-symptomatic stage. In addition, they may be followed over time. In addition, because the present biosensors are sensitive and accurate, ratios of amounts of different antigens may be calculated, e.g. ratio of GAD antibody to insulin antibody.

The present methods use detection antibodies that detect the human antibodies that are selectively bound to the antigens on the array. These may be for example goat anti-human antibodies. They may be specific for subtypes of interest, for example, they may be antibodies specific for detecting anti-GAD IgG2a antibodies. They may be antibody fragments or other markers that are known for specifically binding to human antibodies, in particular human Fc portions of antibodies.

The present assays may be designed in a point-of care device. That is, it may consist of a glass or plastic chip having multiple linear flow channels. The medical sample is prepared by dilution and plasma is separated. The sample is flowed into the chip. As the sample solution flows by diffusion through the channels, the biomarkers will attach to these surface antibody spots. Unbound material is washed off. Then fluorescent labeled antibodies that bind to human antibodies are added to the chip. Different antibody spots are used to allow multiple simultaneous measurements of different biomarkers. The chip is then read with a fluorescent imager that quantifies the amount of biomarker attached to each spot. After reading, the chip is discarded.

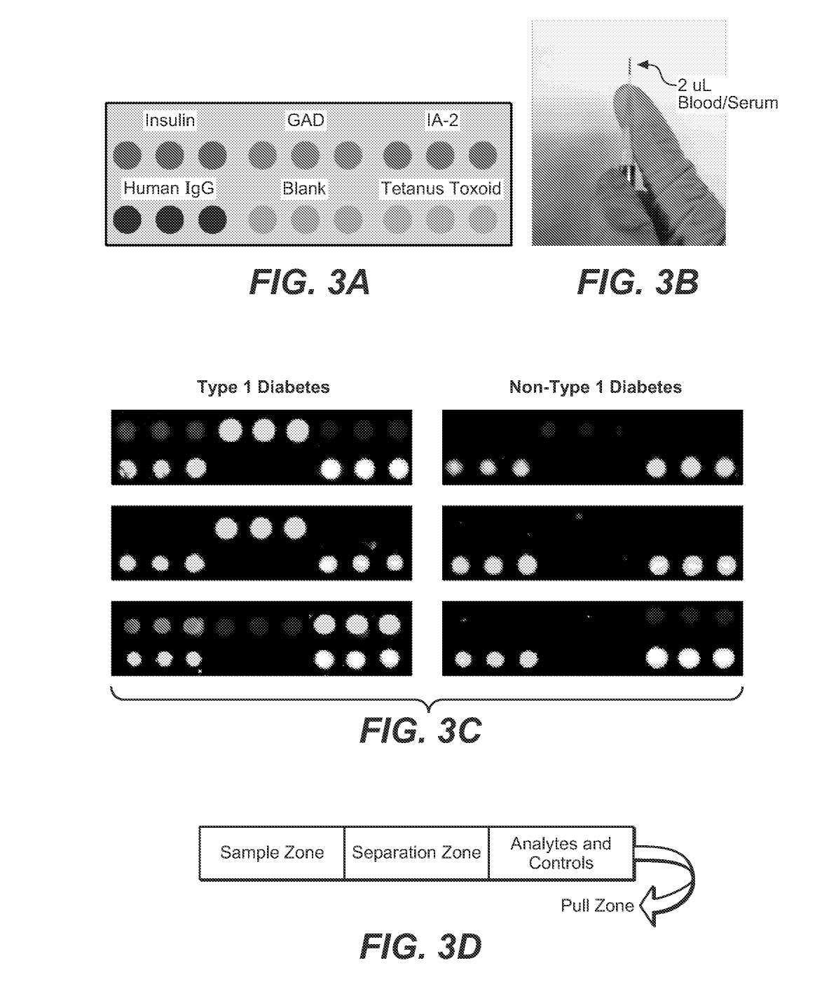

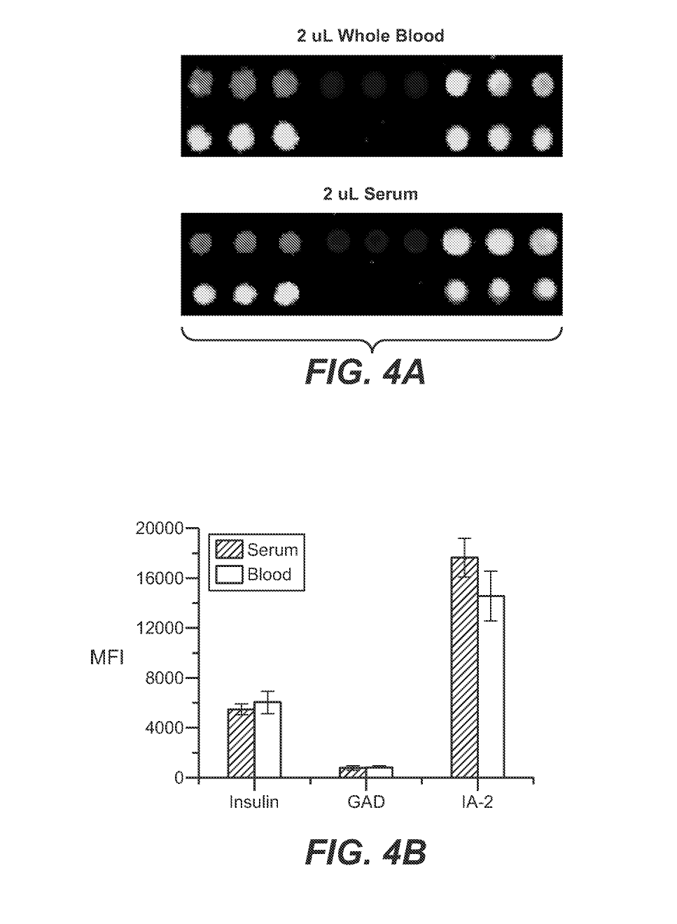

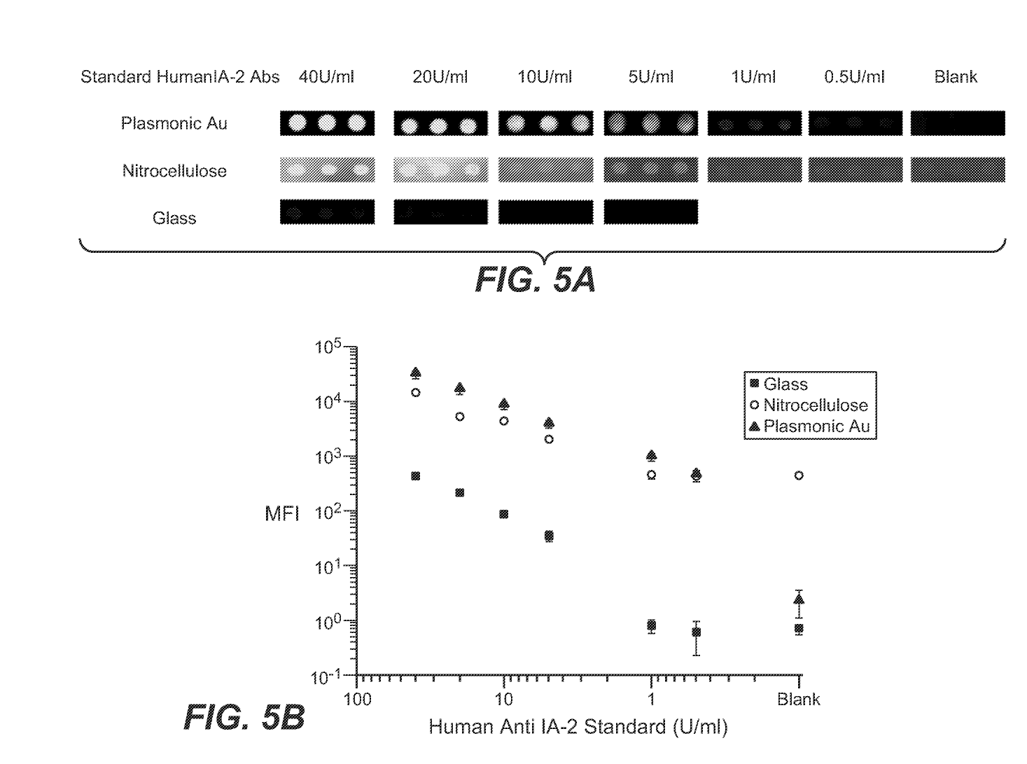

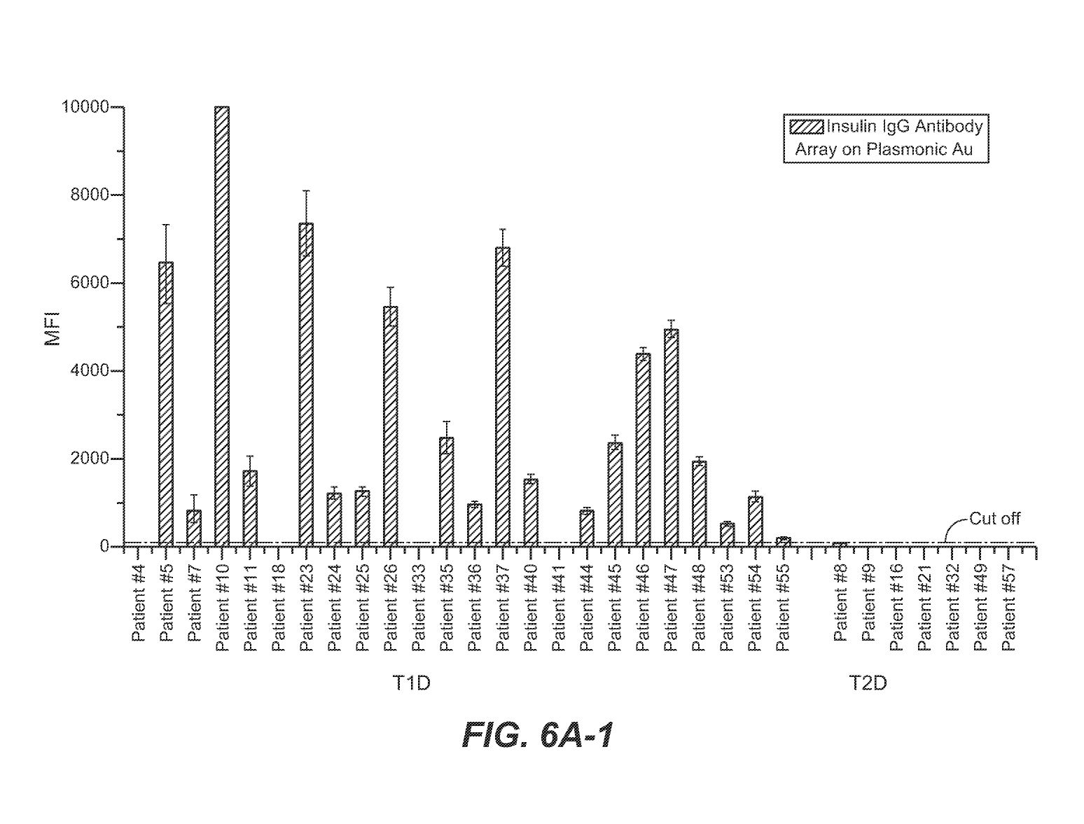

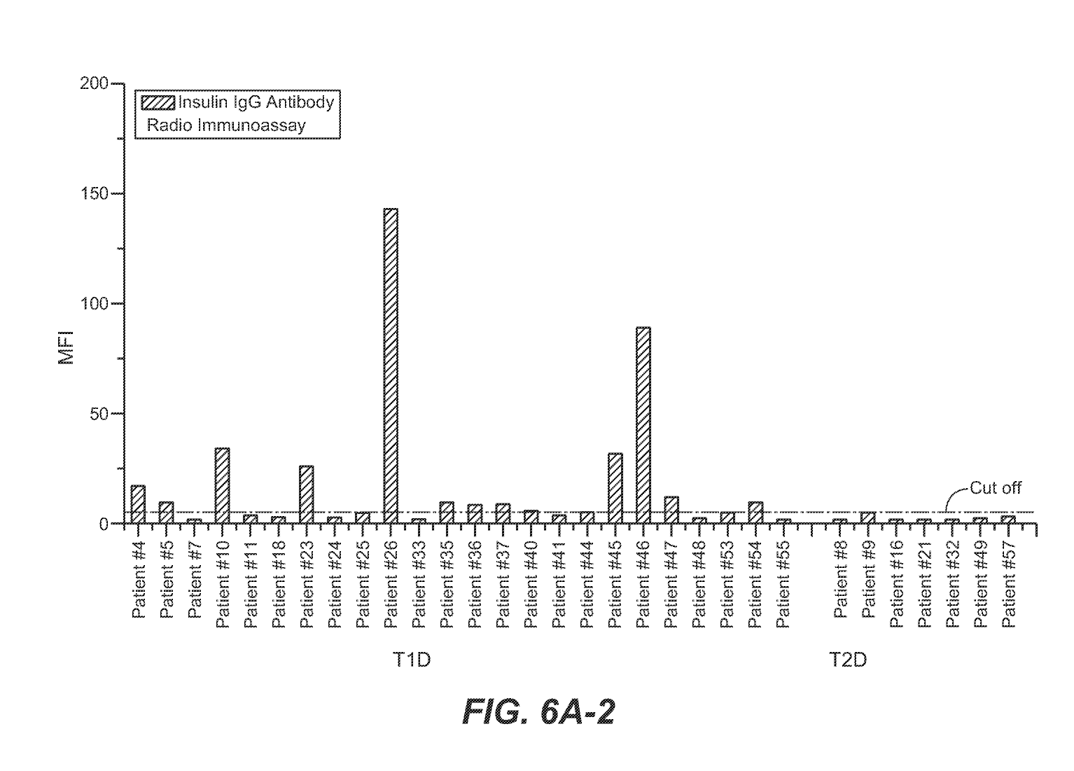

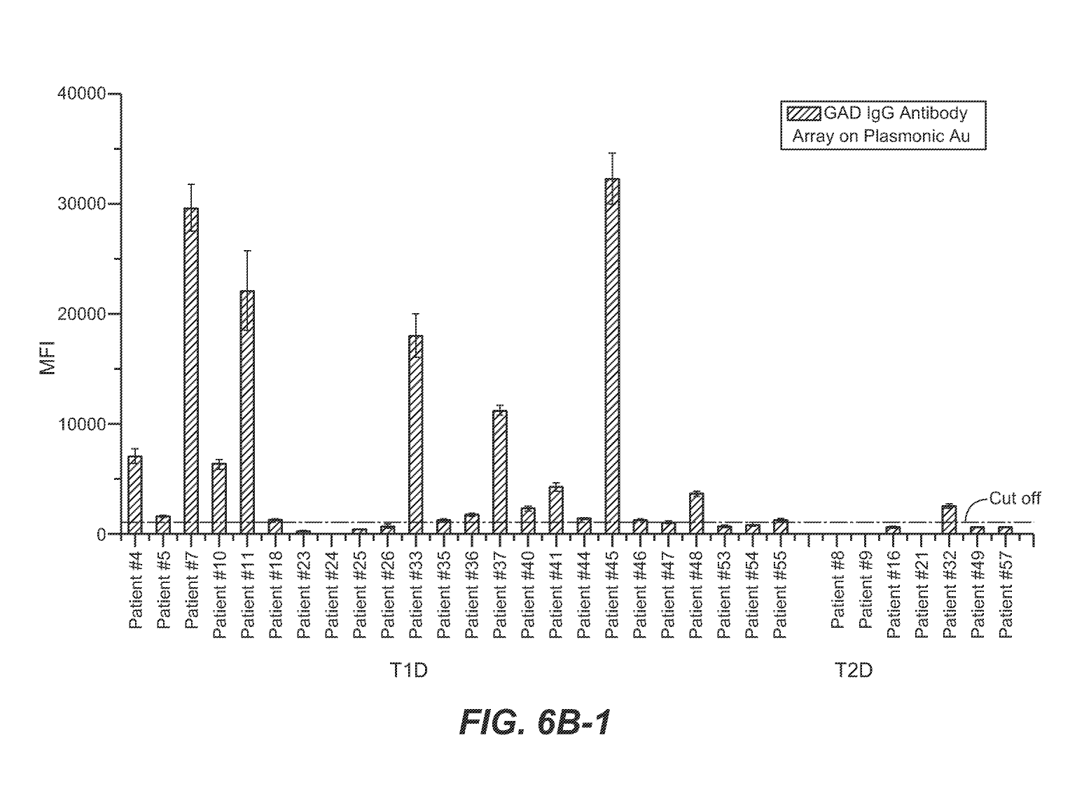

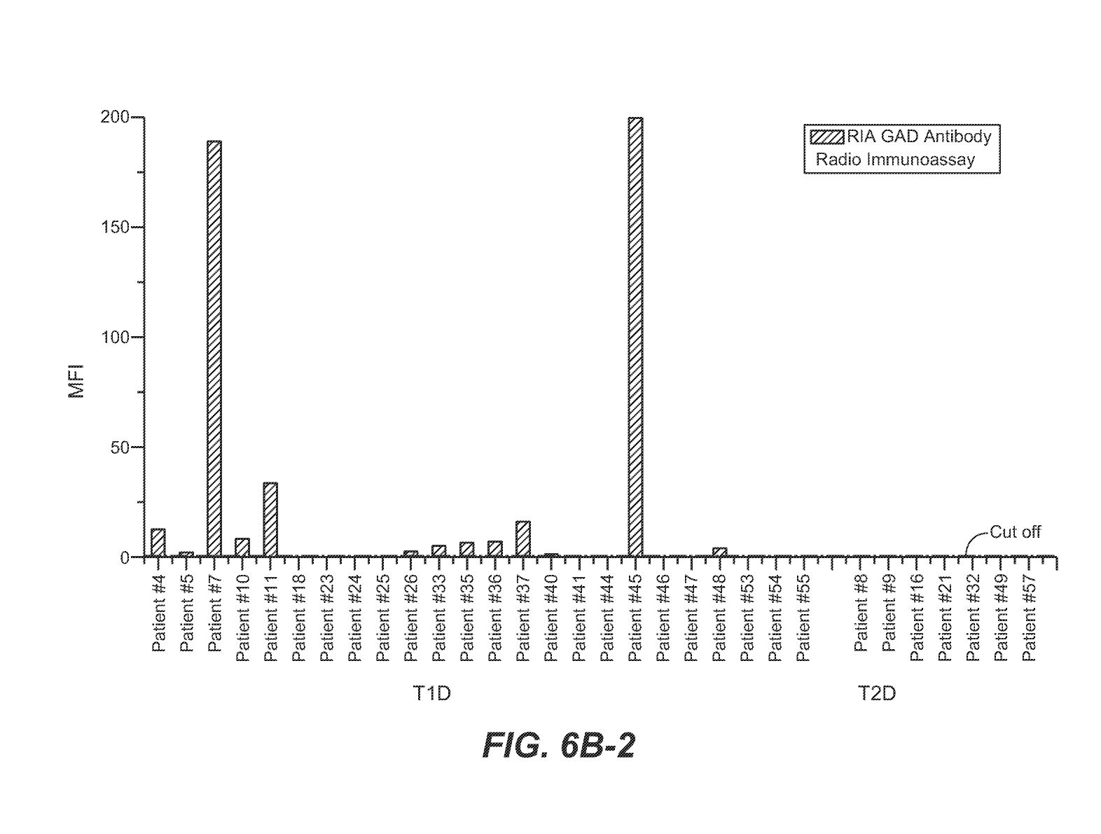

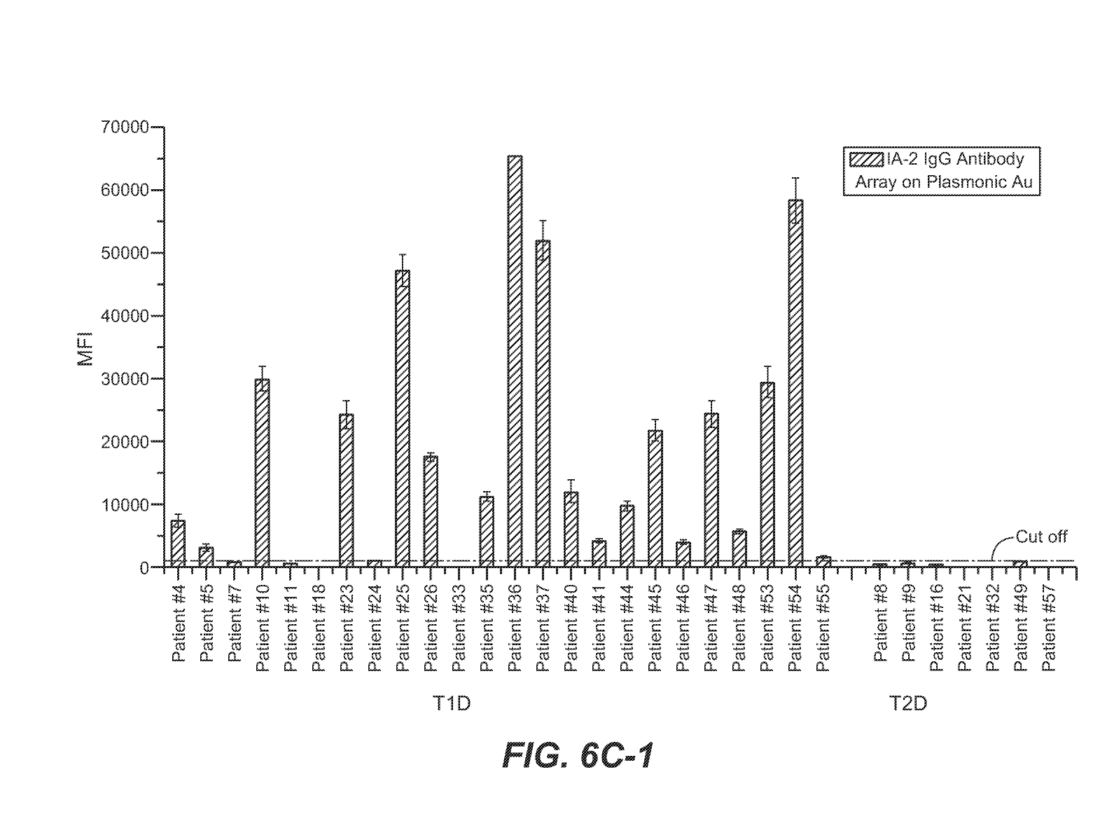

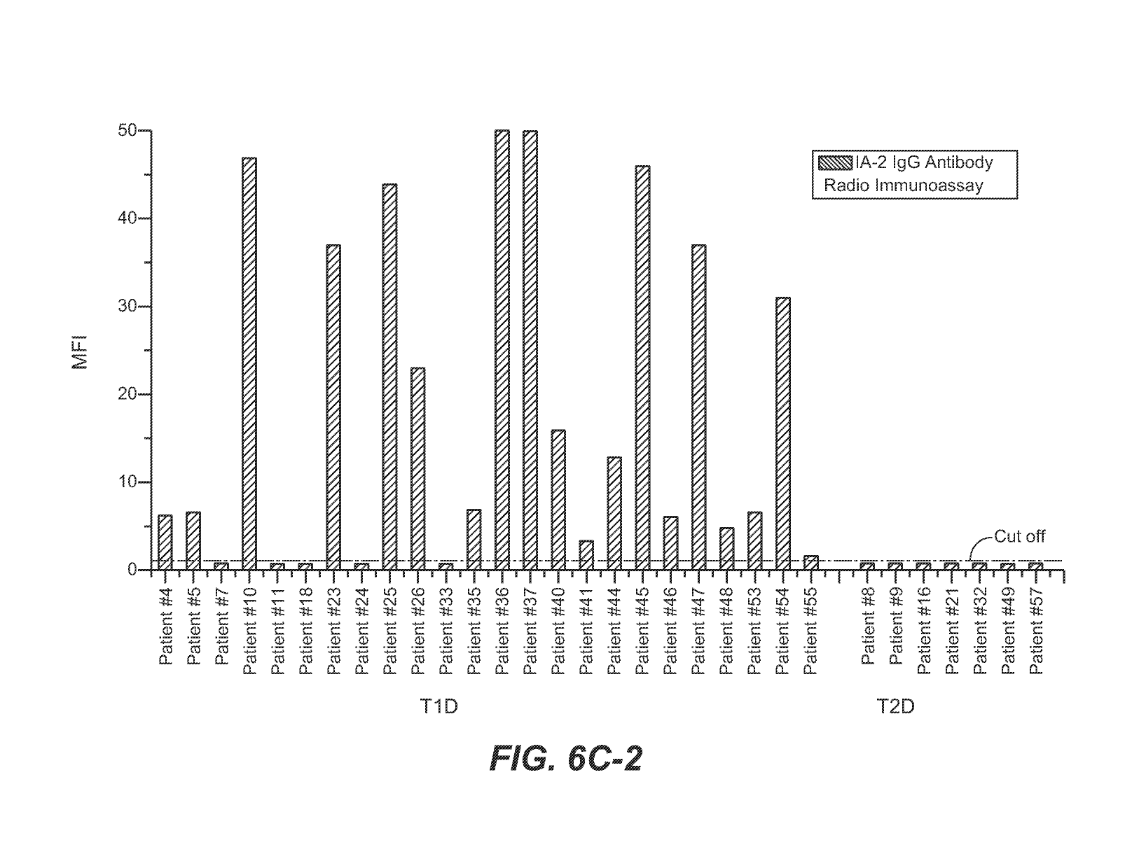

As can be seen in FIG. 3A, antigens are arranged in triplicate spots on the Au surface. IgG antibodies in a patient's serum that bind to these spots are detected and quantified with a microarray scanner. The scanner averages the fluorescence intensity signal over the three distinct spots, and reports this as a mean fluorescence intensity (MFI) for a given patient against each specific antigen. Cut-off values for positivity are established for each marker, e.g. horizontal lines in FIGS. 6A, 6B and 6C for, respectively, insulin, IA-2 and GAD. FIG. 3C shows insulin negative, GAD positive and IA-2 positive (top row and second row, left); and insulin and IA-2 positive (bottom row). As can be seen there, patients who are negative for a certain autoantibody exhibit MFI that are several orders of magnitude below the lowest positive signal. The cutoff as shown in the figures was established by taking the mean value of the T2D signal from the plasmonic Au plus 2 standard deviations. Clinically, an endocrinologist only cares whether a patient has a positive or negative signal, and the degree of positivity of this signal is insignificant. Additionally, only one autoantibody has to be positive in a symptomatic patient to declare type 1 diabetes, and it does not matter which one. It can thus be seen that the plasmonic Au assay picked up positive patients that were missed in the RIA assay.

It is believed that that degree of positivity and diabetes autoantibody profile are clinically relevant, and that this will become more evident once clinicians have a platform that is easy and affordable enough to allow detection and tracking of diabetes autoantibodies. It is thus contemplated that quantitative data can be determined from different markers, using the present assay. For example, a ratio of GAD to ICA512 to insulin antibody titers (or MFI numbers) can be determined. Additionally, there have been recent advances in drugs used to prevent progression to insulin dependence in individuals who are known to be diabetes autoantibody positive, including in those who do not yet have diabetes. The present assay will be useful in the success of such interventions in a given individual over time. In addition, the present platform will allow accessibility in every clinical environment with ease that may one day facilitate general population screening to identify those who will benefit from prevention strategies.

The microarray scanner to be used with the present plasmonic Au chips has a similar capability as the current microarray scanner that detects a signal as “positive” or “negative,” and that also quantifies the “positive” signals with a specific MFI numerical value. The reader may be small and portable, and be either battery or solar powered. A touch screen with multiple-language capability will allow accessibility across countries and across different skill sets of health care providers. A paper printout of the result (similar to current urinalysis readouts) may facilitate areas with paper charting for a given individual over time.

The present microarray can be provided on a microfluidic chip. The chip is designed, as shown in FIG. 3D, to have a sample zone where a blood sample will be applied (ideally a fingerstick drop of blood, and the chip may incorporate a built-in fingertip lancet device). The blood sample will be pulled through microfluidic channels toward the opposing end of the chip by a force such as capillary flow or potential energy from a vacuum channel. The blood will pass across a separation zone where serum (containing the autoantibodies) will be separated from the other whole blood components that will remain behind. The serum will next pass through the analyte zone where the antigens will be coated on the Au surface as distinct serial or parallel bands; IgG that recognizes a given antigen will become anchored in the analyte zone. The remaining IgG/serum will flow across the control zone, where tetanus toxoid (currently purchased from Santa Cruz Biotechnology) will be coated on the Au surface as a distinct band (children and adults in this country are generally immunized against tetanus toxoid and should have IgG against tetanus toxoid as early as six months of age). This control zone will confirm the validity of a given test and reflect ability to detect specific IgGs. Additionally or alternatively, anti-human IgG is applied to the at the control zone to increase applicability to non-immunized children including in the developing world. After the serum has been wicked/pulled to the pull zone, we will add a fluid reagent of anti-human IgG antibodies labeled with near-infrared fluorophore(s) near the sample zone or separation zone. This reagent solution will also be pulled toward the pull zone, and will bind to any IgG antibodies that were anchored at the analyte zone. Once this solution has reached the pull zone, the chip is read, ideally with a small/inexpensive/portable reader that detects and quantifies the mean fluorescence intensity (MFI) of the near-infrared fluorophore(s) at each band of the analyte and control zones.

This chip-based technology in not only suitable for simultaneous detection of IgG, IgM and IgA antibodies against the type 1 diabetes antigens, but from the same blood sample, can likely also detect antibodies specific to other autoimmune diseases known to have increased incidence in the type 1 diabetes (T1D) population including IgG against thyroid peroxidase, IgG against thyroglobulin, and IgG against TSH receptors (helps to assess for Hashimoto's hypothyroidism and Graves' hyperthyroidism), IgA against tissue transglutaminase and IgG against deamidated gliadin peptide (helps to assess for Celiac disease), and IgG against 21-hydroxylase and IgG against side chain cleavage enzymes (helps to assess for Addison's disease).

Suitable designs for microfluidic chips may be adapted from, e.g. designs shown in Morgan et al. US 2005/0074900, “Microfluidic flow-through immunoassay for simultaneous detection of multiple proteins in a biological sample”, published Apr. 7, 2005, Swee Chuan Tjin et al, “Microfluidic immunoassay device,” U.S. Pat. No. 8,158,363, etc. Suitable designs for NIR microfluidic chip readers may be adapted from, e.g., Guilfoyle et al., “Optical system enabling low power excitation and high sensitivity detection of near infrared to visible upconversion phoshors,” US 20120280144, Cyr et al., “Scanners for reading near infrared fluorescent marks, U.S. Pat. No. 5,959,296, etc.

### EXAMPLES

### Example 1

### Plasmonically Active Au Substrates

Plasmonic nanostructured gold substrates are fabricated, and are capable of optimal fluorescence enhancement up to 1000 fold for NIR fluorophores, as described in the above-referenced patent application and publication by Tabakman et al. Chemical synthesis and lithographic patterning techniques are used for the formation of plasmonic gold films on glass capable of maximizing the fluorescence enhancement of NIR dyes coupled to antibodies to the diabetes autoantigens in a patient sample. The plasmonic resonance is controlled by the gold nanostructure patterns to match to emission of NIR fluorophores. Nano-gaps between gold nanostructures are tuned to maximize local electrical field enhancement for optimal NIR-FE for fluorophores such as cy5, cy5.5, Atto 647N, cy7, and IR800.

### Example 2

### Detection of Diagnostic Biomarkers

(Taken from copending Ser. No. 13/728,798). This example employs protein microarray printing onto a gold plasmonic substrate on a glass chip as described in the examples above. This array allows detection of autoantibodies in a patient sample. The antibodies associated with diabetes type I are raised against small volumes of antigen. The present array and use of the plasmonic substrate with enhanced near-infrared fluorescence provides significantly improved sensitivity, as the gold film amplifies the fluorescent signal labels on detection antibodies. Additionally, this platform allows for multiplexed testing for more than one diabetes autoantibody from a single patient sample, in addition to testing for diagnostic autoantibodies for other autoimmune diseases known to have increased prevalence in patients with type 1 diabetes from the same sample (including celiac disease, autoimmune hypothyroidism, and Addison disease).

### Example 3

### Surface Chemistry for Applying Antigens for Effective Antibody Capture

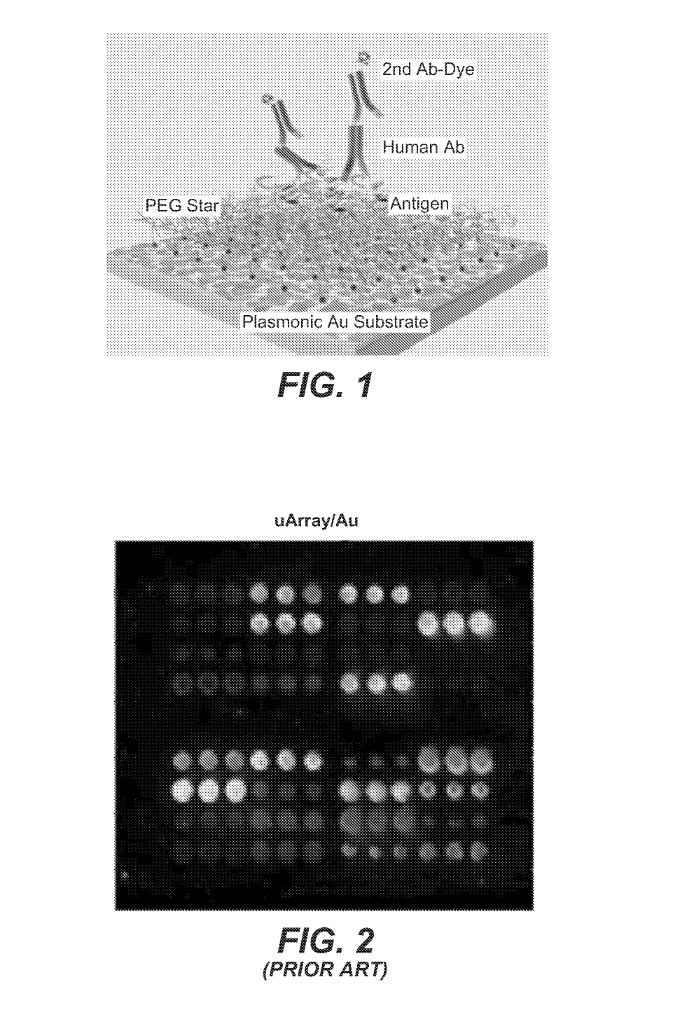

While fluorescence-enhancement will enhance positive binding signal, effective surface chemistry on Au plasmonic substrates is used herein to immobilize capturing antibodies and prevent non-specific binding, thus optimizing positive binding and reducing background and false signal. We here modify the plasmonic gold film for immobilization of multiplexed protein probes by first making self-assembled monolayers (SAM) of thiol-containing molecules terminated with carboxylate groups. Branched hydrophilic polymers of 6-arm-poly(ethylene glycol) (PEG)-NH2 is then grafted to SAMs, followed by reaction with succinic anhydride to obtain carboxylic acid groups off the 6-arm-PEG for protein immobilization. This method is surprisingly advantageous, since it employs branched-PEG star polymers as a hydrophilic, biocompatible cushion (FIG. 1) on Au to allow oriented immobilization of protein probes with minimal conformational perturbations and denaturing, and to minimize false signals due to the stronger ability of branched PEG in preventing non-specific binding effects than linear PEG chains. See, for comparison, Chen Z, Tabakman S M, Goodwin A P, et al. Protein microarrays with carbon nanotubes as multicolor Raman labels. Nature Biotechnology 2008; 26:1285-1292, and Liu Z, Davis C, Cai W B, et al. Circulation and long-term fate of functionalized, biocompatible single-walled carbon nanotubes in mice probed by Raman spectroscopy. Proceedings of the National Academy of Sciences of the United States of America 2008; 105:1410-1415.

The insulin-related antigens as applied in the examples are commercially available. Specifically, these are: insulin, regular pharmaceutical insulin; GAD65 antigen, purchased from Kronus, Inc. Boise, Id.; and IA2 (ICA512) also purchased from Kronus, Inc.

### Example 4

### Testing of Subjects for Diabetes-Related Antibodies

Following optimization of the surface chemistry on the Au platform, multiplexed native structure insulin, GAD65 and IA-2 antigens are deposited on a slide as triplicate dots. The as-prepared slide is exposed to serum and whole blood collected from a patient suspected of having a diabetic or pre-diabetic condition. For experimental purposes, one may test insulin-naïve type 1 and type 2 diabetics who have quantified RIA data from the Stanford pediatric diabetes practice. Based on the below example, samples are diluted 1:10 with fetal bovine serum and introduced to the surface for 20 minutes. We then wash and expose a secondary anti-immunoglobulin conjugated with a NIR dye for 20 minutes. Following a final wash step, we assess the slide with a microarray scanner and quantify signal as mean fluorescence intensity (MFI).

Three diabetes autoantibodies are detected, based on the antigens ICA512, Insulin and GAD65. Autoantibodies to insulin and GAD 65 were detected in test subjects and the results confirmed by RIA.

Data obtained from the present plasmonic substrates is compared to RIA for each diabetes autoantibody. Mean fluorescence intensity (MFI) values from subjects known to have a clinical diagnosis of T2D are used to determine their MFI mean plus two standard deviations to declare a positivity cut-off. The sensitivity and specificity of each autoantibody test will be analyzed as receiver-operating characteristic (ROC) plots [Zweig M H, Campbell G. Receiver-operating characteristic (ROC) plots: a fundamental evaluation tool in clinical medicine. Clin Chem 1993; 39:561-577]. As an adjunct, MFI quantification for the three autoantibodies can be converted using a calibration curve derived from spiked serum sample, and compared to the RIA values. Correlation between assays is examined with scatter-plots and linear regression analysis, and agreement is assessed using Cohen's kappa measurement. Exemplary data for over 20 patients suspected of having T1D and T2D are shown in FIGS. 6A-1, 6A-2 (insulin Ab), 6B-1, 6B-2 (GAD Ab), 6C-1, and 6C-2 (IA-2 Ab).

### Example 5

### Probing Condition Optimization of Human Serum

We demonstrated that a 1:10 dilution of human serum in whole FBS improves signal-noise ratio of the microarray by vastly decreasing background caused by non-specific binding (NSB). The background signal in a.u. was around 40,000 for whole serum, around 5,000 for 1:1 FBS, and near zero in 1:10 FBS (data not shown)

### Example 6

### Use of Plasmonic Chips on Clinical Samples

Gold Plasmonic Chip Enables Detection of Islet Antigen Autoantibodies

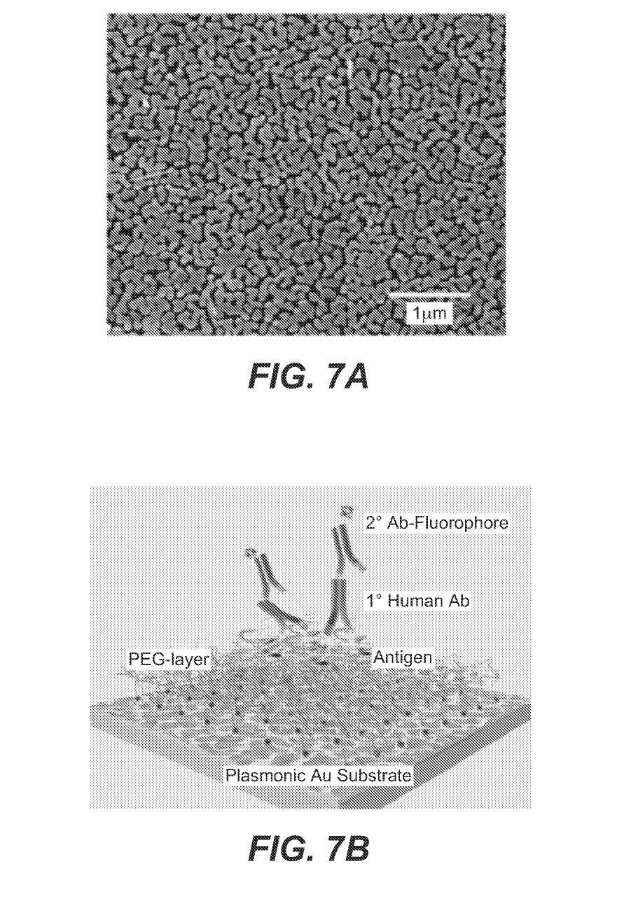

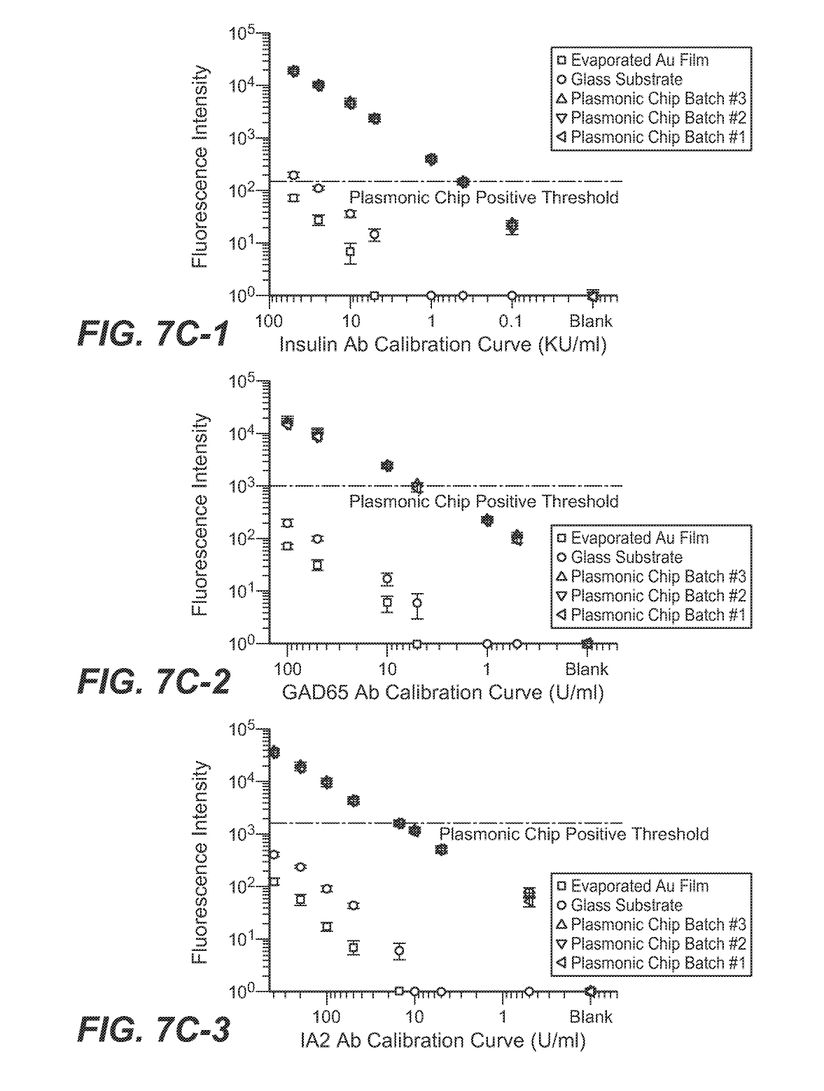

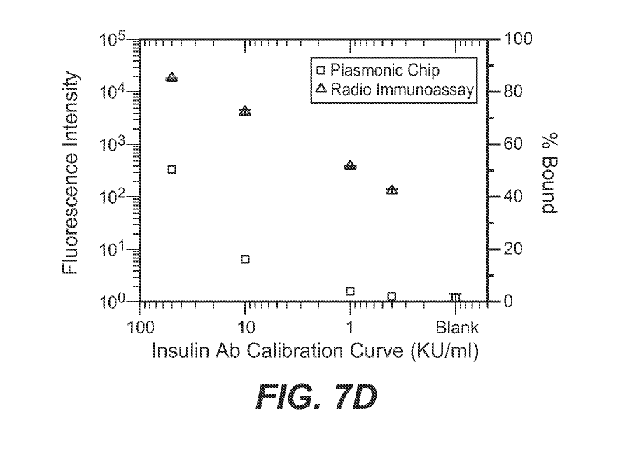

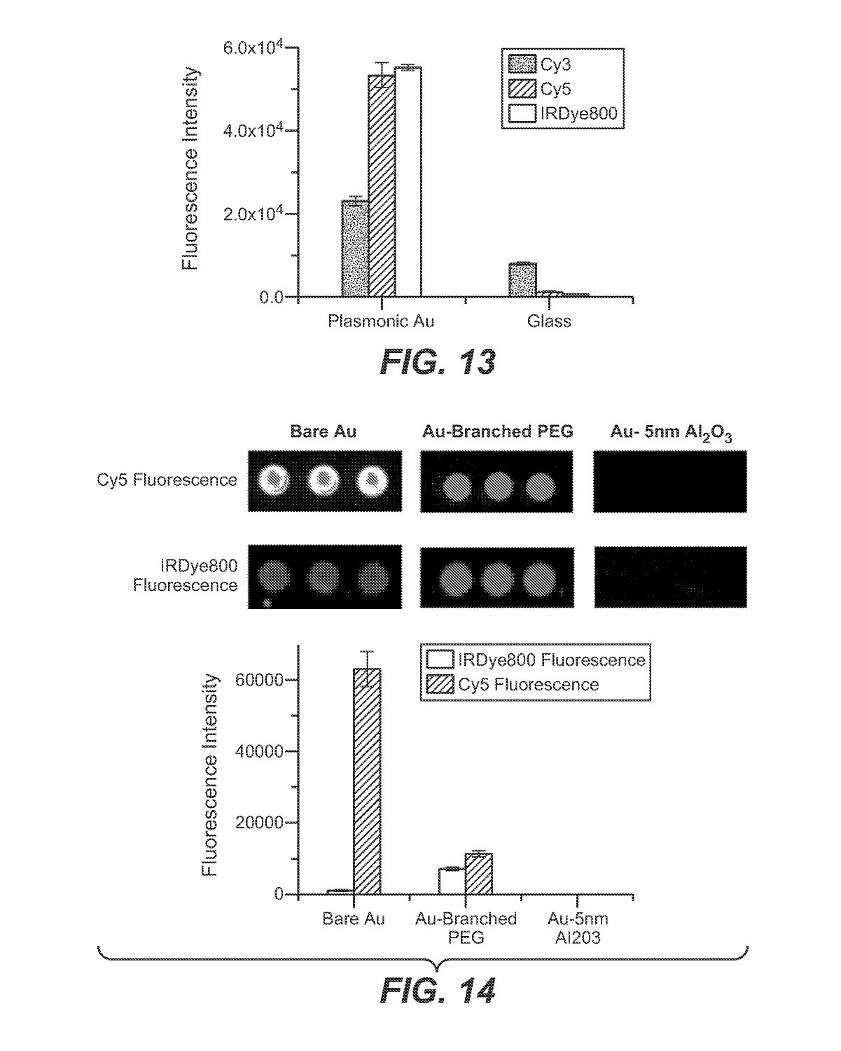

Multiplexed islet antigen microarrays using nanostructured gold islands on a glass surface are shown in FIG. 7A). The gold islands ensure the generation of abundant nanogaps (˜10 nm range) that support electric field enhancement and surface plasmonic resonance to afford near-infrared fluorescence enhancement (NIR-FE) by ˜100 fold (FIG. 7A). Additionally, a biocompatible branched PEG layer was applied over the gold islands to help preserve the native conformation of the islet antigens (FIG. 7B) [see discussion of PEG in Roberts, M. J., Bentlye, M. D. & Harris, J. M. Chemistry for peptide and protein PEGylation. Adv. Drug Deliv. Rev. 54, 459-476 (2002).]. To form a sandwich assay on this platform for islet autoantibody quantification, target islet antigen was immobilized on the plasmonic chip, probed with human serum (to capture specific autoantibodies against the target antigen) and then detected with IRDye800 labeled anti-human IgG antibody. The abundance of specific autoantibody in the serum was reflected by the measured intensity of IRDye800 fluorescence (FIG. 7C). A calibration curve for insulin, GAD65 or IA2 autoantibody was first obtained on a plasmonic chip using commercial reference samples containing known concentrations of the autoantibody. We observed 2-3 orders of magnitude increase in sensitivity on the gold substrate compared to glass. Importantly, calibration titration curves exhibiting <10% coefficient variation (CV) were obtained with different batches of plasmonic chips in independent experiments even at low concentrations of autoantibodies, down to 0.1 U/mL (FIG. 7C), demonstrating the high reproducibility and consistency of the plasmonic gold substrates. Note that, in contrast to the plasmonic gold substrates, vacuum evaporated smooth gold films produced a fluorescence quenching effect, instead of enhancement, resulting in a decrease in sensitivity compared to traditional glass substrate (FIG. 7C). Other microarray substrates, such as glass, nitrocellulose or smooth gold, had significantly lower sensitivity and specificity than the plasmonic gold surface (FIG. 13). Of note, compared to RIA, the plasmonic gold platform had better sensitivity for detecting insulin autoantibodies (FIG. 7D).

Detection of Islet Antigen Autoantibodies in Ultralow Volumes of Human Serum and Blood

In addition to improved sensitivity, the use of this plasmonic chip for diabetes diagnostics has several additional advantages over RIA (Table below), including the ability to use ultralow sample volumes.

TABLE 1
Comparison of the T1D, T2D and non-diabetic subject groups.

| | T1D, N = 26 | T2D, n = 13 | non-diabetic, n = 5 |
|---|---|---|---|
| Gender (M or F) | 14 M, 12 F | 8 M, 5 F | 3M, 2F |
| Age (y) | 9.7 ± 3.8 | 14.1 ± 2.4 | 8.0 ± 3.3 |
| | (1.9-19.5) | (10.2-18.2) | (3.9-13.2) |
| Body Mass Index | 18.0 ± 4.6 | 31.6 ± 9.2 | 19.1 ± 2.6 |
| (kg/m²) | (12.0-29.9) | (18.7-46.7) | (15.3-22.1) |
| Serum Bicarbonate | 16.0 ± 7.2 | 24.5 ± 5.2 | N.D. |
| level (mEq/L) | (<5-30) | (12-32) | |
| Hemoglobin A1c (%) | 11.3 ± 2.2 | 10.0 ± 2.9 | N.D. |
| | (6.7->15) | (6.6->15) | |

Serum Bicarbonate <5 meq/L = 5 meq/L;
Hemoglobin A1c >15% = 15%
Values reported as sample mean ± S.D. (absolute range);
N.D. = not done

A plasmonic chip with a 6-plexed antigen array was constructed for simultaneous multiple islet antigen autoantibody detection with low samples volumes (FIG. 3a). The islet antigens were each robotically printed in triplicate spots onto the branched PEG coated gold film, resulting in a microarray with feature diameters of ˜400 μm. As a technical positive control, we printed human IgG for recognition by the secondary detection antibody that we used. IgG spots are shown in the lower left. As a biological positive control, we printed tetanus toxoid, as immunization to tetanus is part of standard health care practice resulting in protective IgG antibodies in the serum of the majority of the population of the United States. Spots of PBS were used as negative controls.

In order to validate this platform using ultralow sample volumes, we tested volunteer patients with new-onset diabetes who were simultaneously undergoing standard RIA testing for islet antigen autoantibodies.

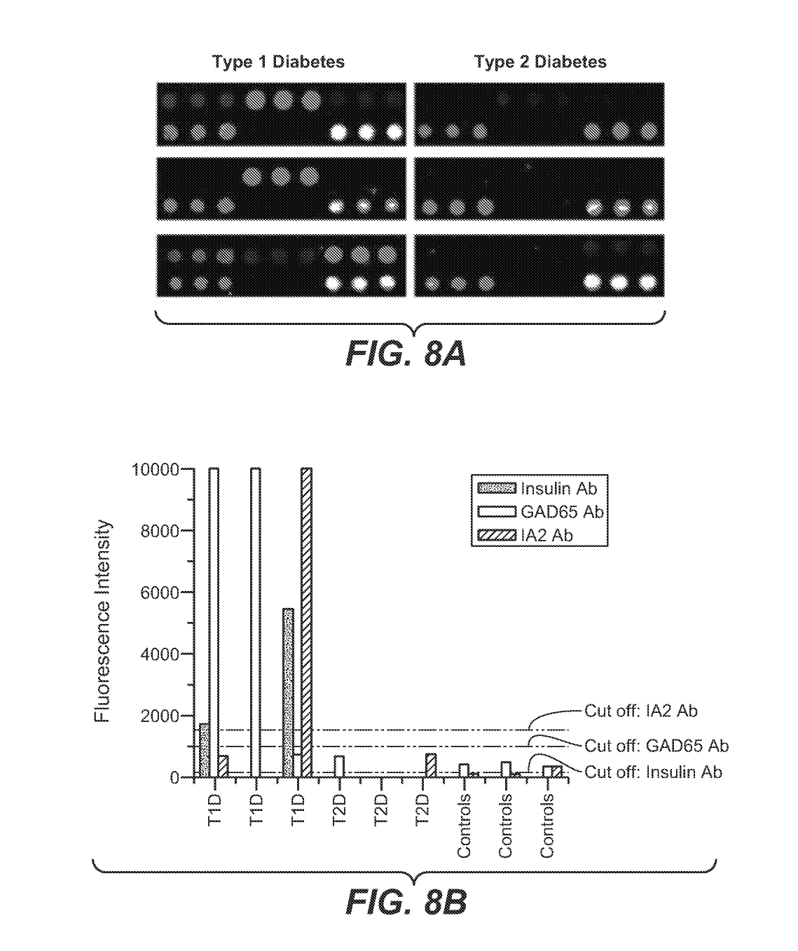

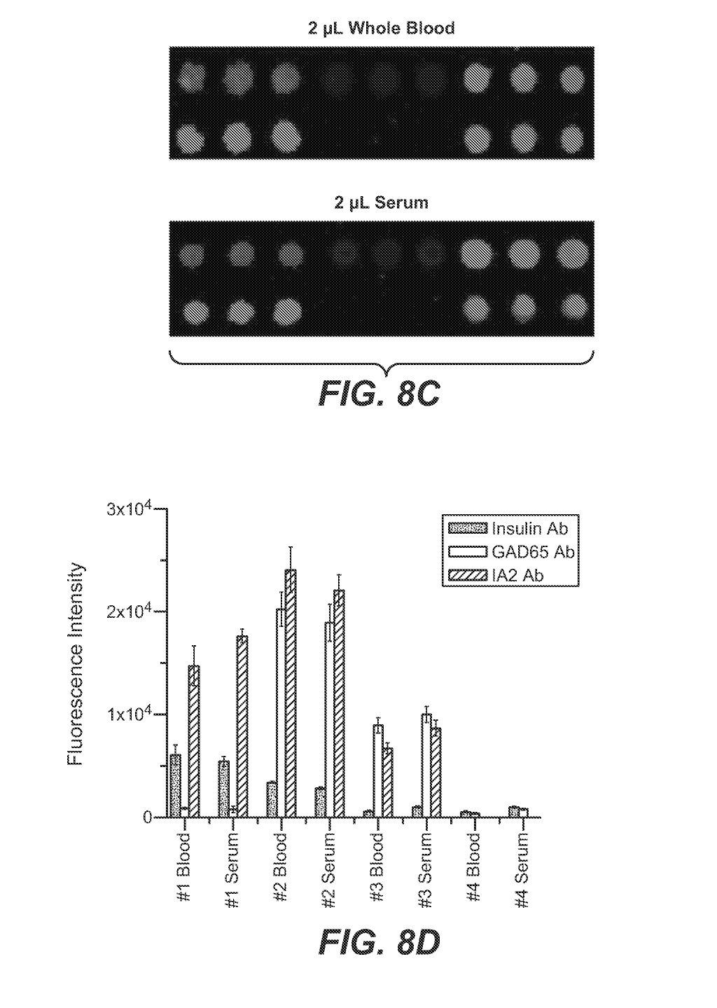

About 2 μL of human serum or blood (described in Example 7—“Materials and Methods”) was diluted 1:10 and used to probe the chips (FIG. 8). After processing, the chips were analyzed in a standard microarray scanner to detect and quantify the signals. For internal quality control, positive signals were always detected on the IgG and tetanus spots and no signal was detected on the PBS spots on every chip tested (PBS can replace “Blank” in FIG. 3A. If a patient's serum contained autoantibodies against one or more islet antigens, a positive signal was detected specifically on the respective antigen spots (FIGS. 8A, 8B). With this technique, we also were able to detect T1D autoantibodies in ultralow volumes of whole blood samples from humans (FIG. 8C, 8D). The outcome of autoantibody detection in whole blood samples was consistent with that in processed serum samples from the same patient (FIG. 8D). The extremely small volume of blood required for our assay indicates that it will be feasible to use finger-prick sampling rather than venous blood draws (FIG. 3B).

Plasmonic Chip has Equal Sensitivity and Specificity as RIA for T1D Diagnosis

We collected samples from patients with new-onset diabetes and non-diabetic controls in our Pediatric Endocrinology Clinic and research center at Stanford Medical Center. We excluded any subject/guardian not willing or able to give informed consent, and those known to have conditions such as active bleeding, severe anemia or other medical conditions whereby removal of an extra tube of blood would increase risk to the subject. Additionally, we excluded any subject not considered to be insulin-naïve (defined as anyone previously treated with insulin for more than three days) as exogenous insulin injected subcutaneously is thought to result in non-pathologic antibody production against this protein. We collected samples from 26 patients with new-onset T1D, 13 patients with new-onset T2D and 5 non-diabetic controls. The patient characteristics are summarized in Table 1 with statistical analysis summarized in the Example 7.

In order to establish positive signal intensity cutoffs on the plasmonic chip, we tested spiked serum control samples containing autoantibodies at the cutoff concentration to determine their mean fluorescence intensity (MFI) equivalence on our platform (FIG. 7C). To facilitate direct comparison of the described assay with the currently used standard, an aliquot of the same blood samples used on the plasmonic gold chip was tested using RIA in a commercial laboratory for every diabetic patient. The results of both assays were compared to the ultimate clinical diagnosis determined by disease progression and insulin requirement six months after the initial presentation (see Example 7). One T1D subject had GAD65 autoantibody testing performed on a different RIA platform and one T2D subject had insulin and GAD65 autoantibody testing performed on a different RIA platform and these values were not included for statistical analysis; omitting these values did not improve the results.

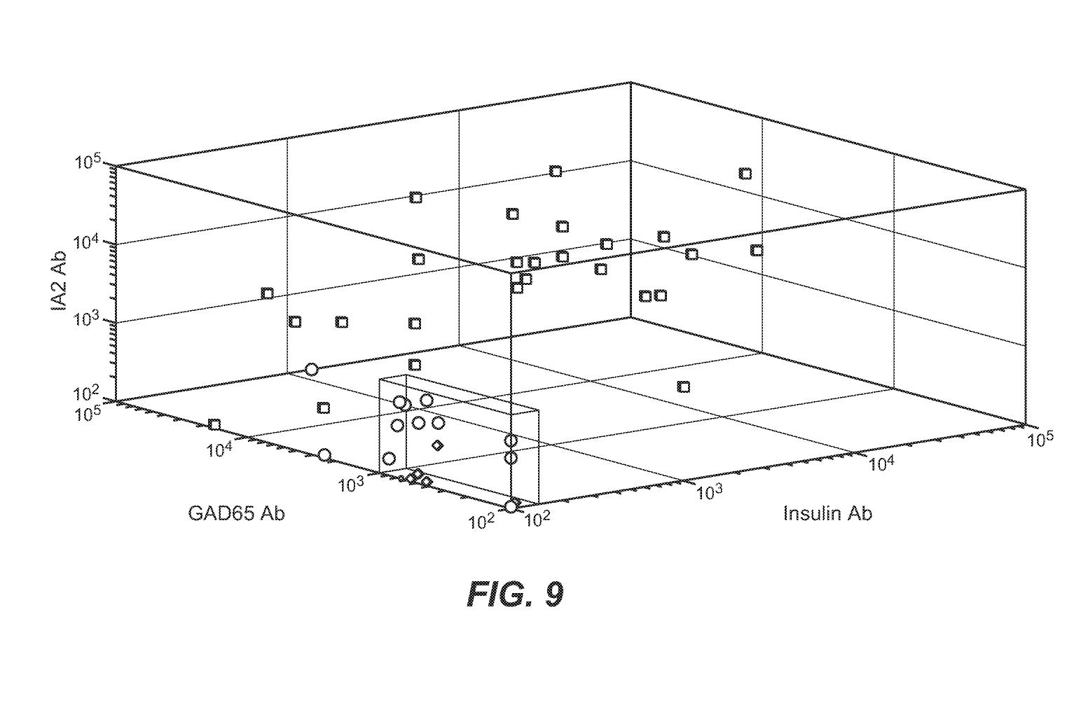

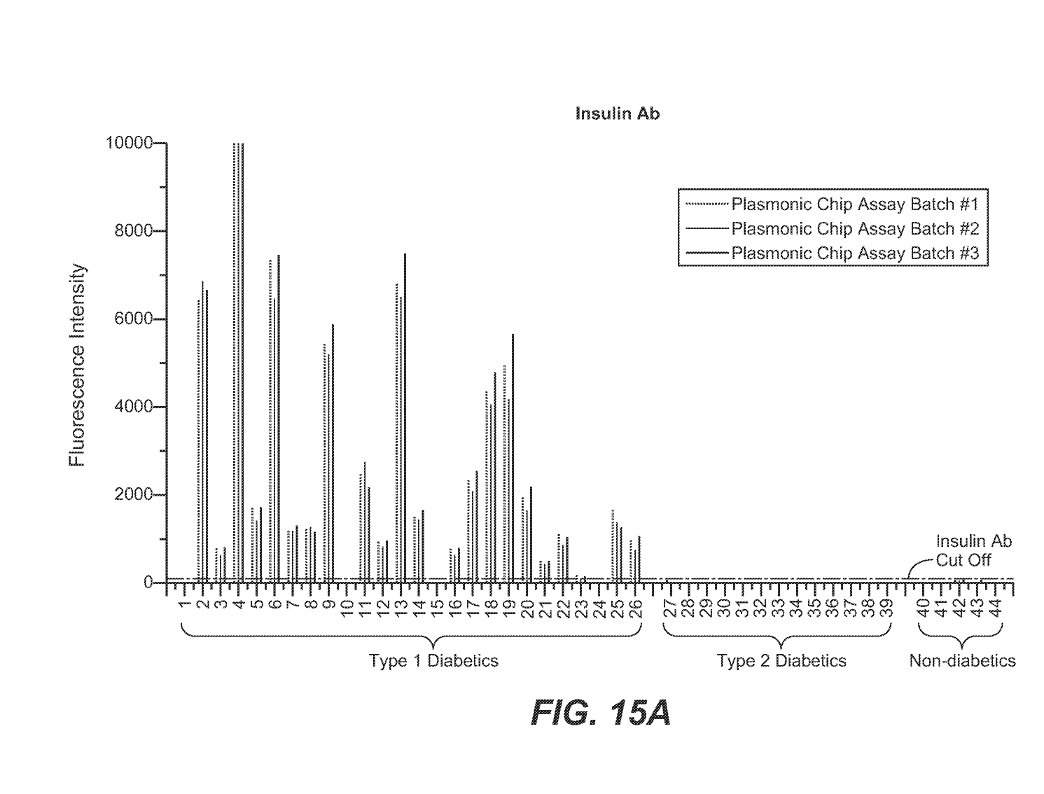

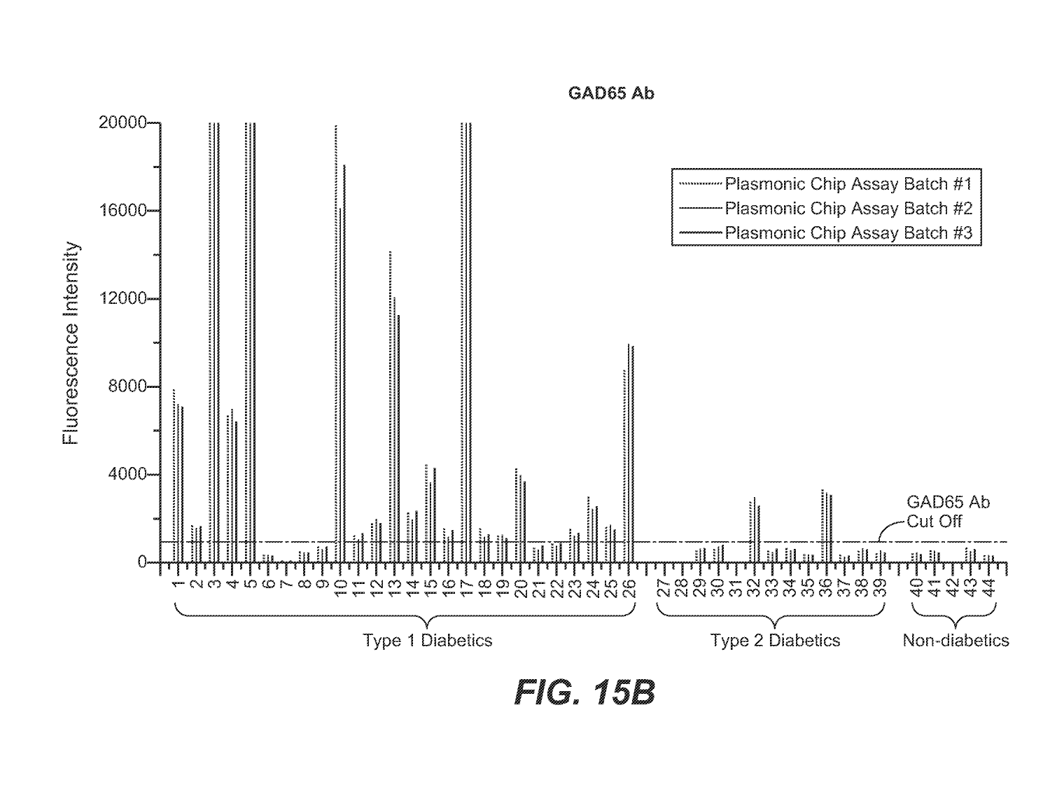

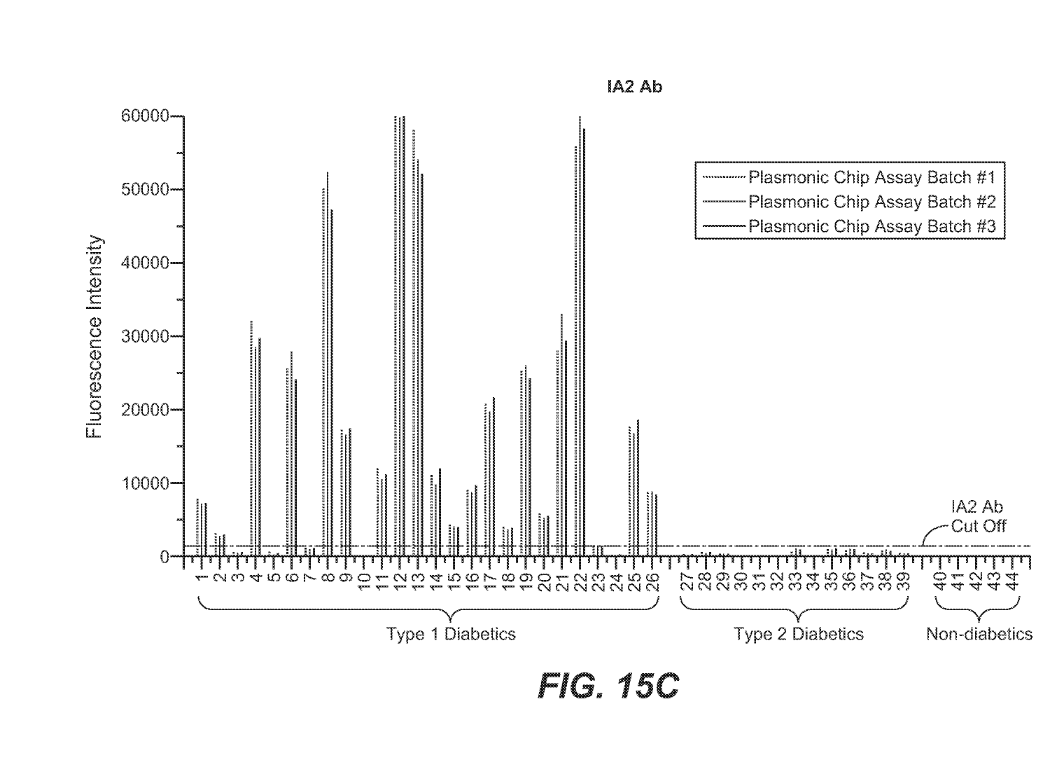

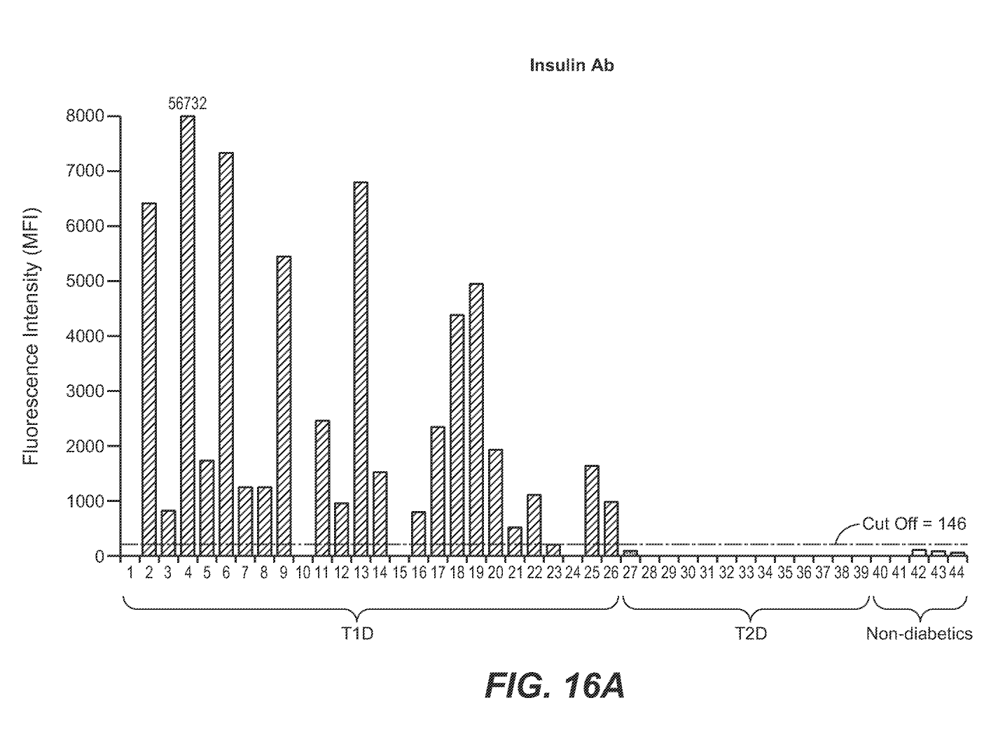

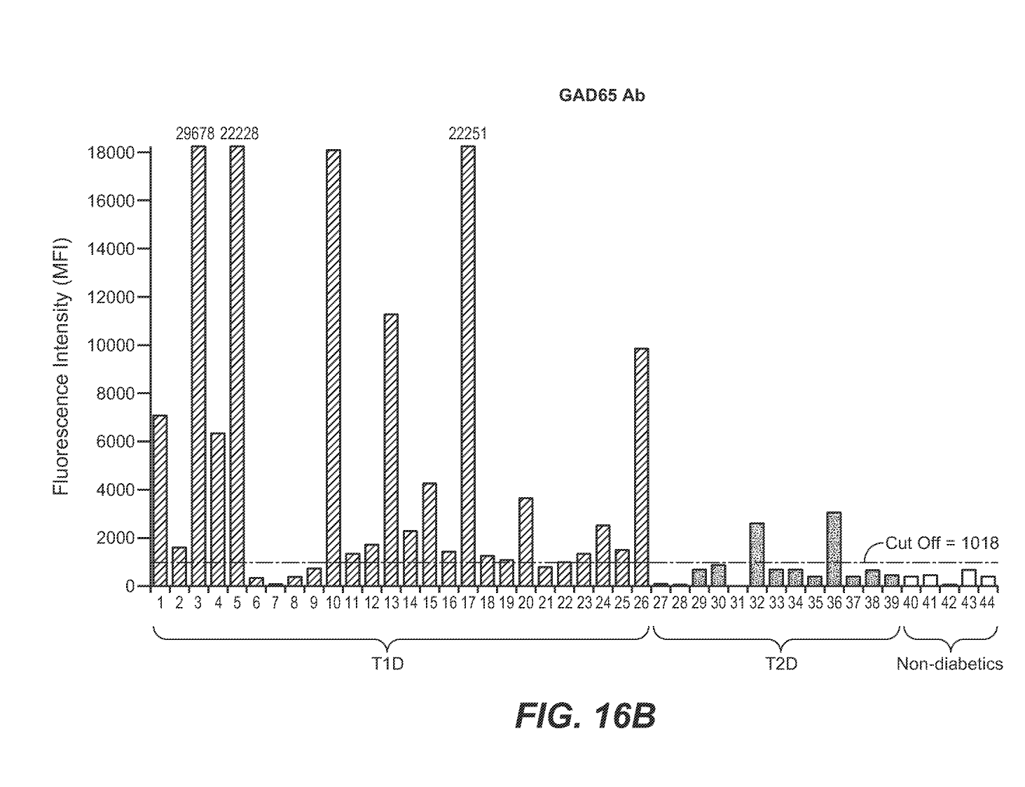

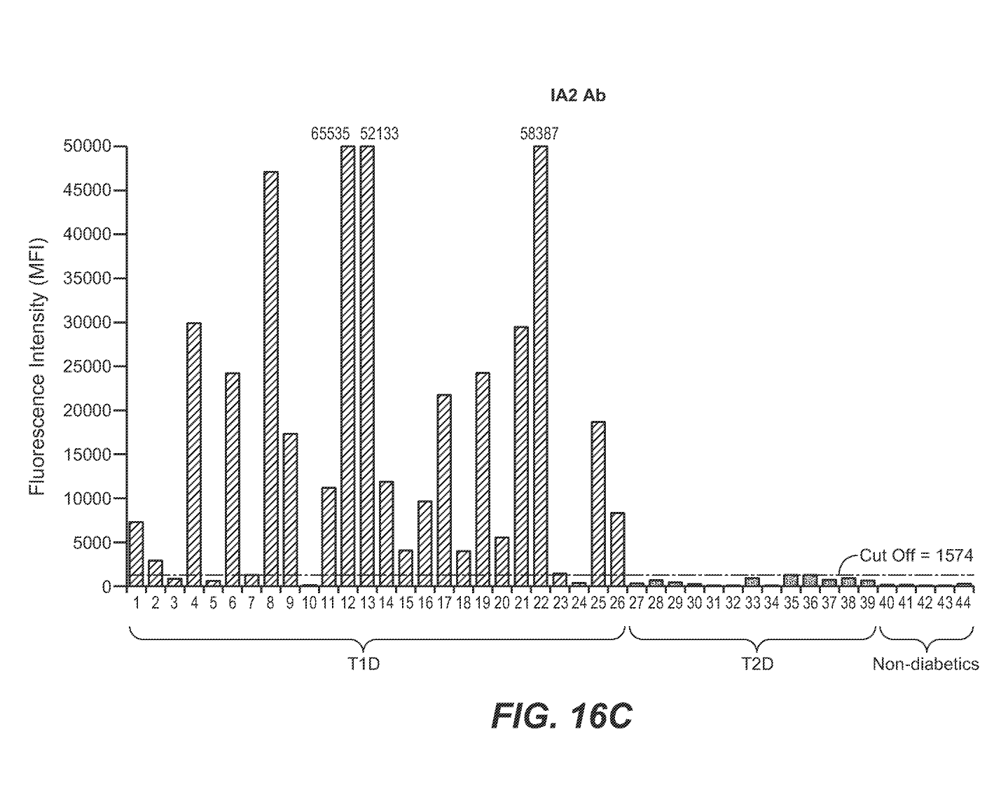

As expected, all of the non-diabetic controls tested negative for all 3 autoantibodies on the plasmonic gold chip platform (FIG. 9 and FIG. 15). Samples from all the diabetic patients were tested for each autoantibody 3 different times, on different days, using different plasmonic chips, with excellent reproducibility (FIG. 15). The mean titers for each autoantibody for every patient are plotted on the same graph in FIG. 9 and individual autoantibody graphs in FIG. 16. We found that the plasmonic gold chip and RIA both have a sensitivity of 100% and specificity of 85% in the patients tested (Table 2).

TABLE 2
Comparison of patient test results by RIA and
Gold Plasmonic Platforms.

| | | |
|---|---|---|
| T1D Sensitivity | 100% | 100% |
| T1D Specificity | 85% | 85% |
| Number of patients with: | | |
| Zero Ab positive | 11 | 11 |
| One Ab positive | 7 | 5 |
| Two Abs positive | 7 | 9 |
| Three Abs positive | 14 | 14 |

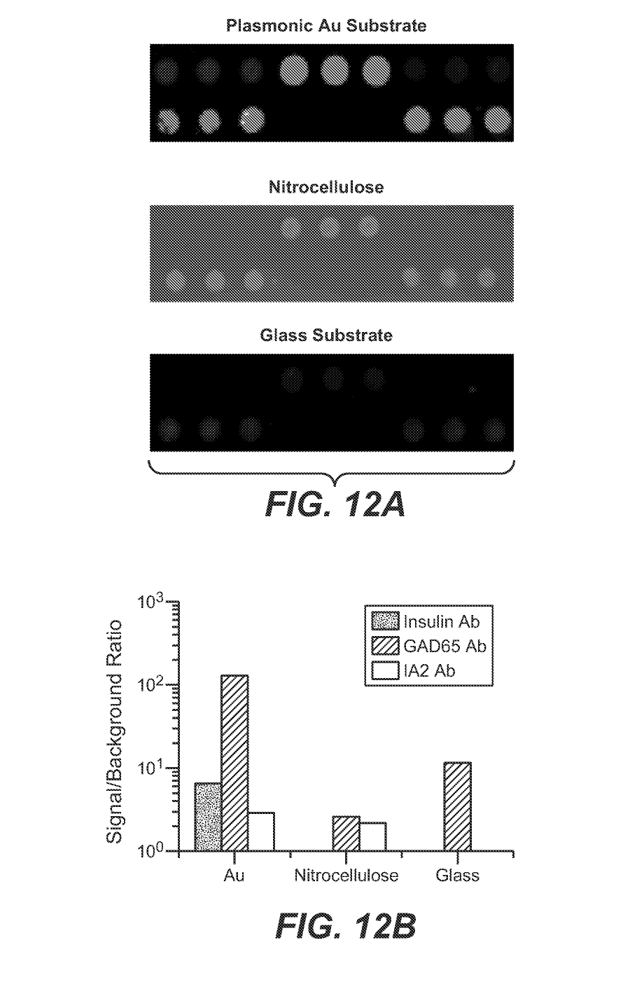

The same serum samples tested on the plasmonic gold chip were also evaluated on microarrays formed on glass and nitrocellulose. We found that the NIR fluorescence signal-to-noise ratios for the detection of islet antigen autoantibodies were far superior on the plasmonic platform (FIG. 12 and Table 1).

Plasmonic Chip Allows Multi-Color Fluorescence Enhanced Detection of Potentially Novel and Earlier Biomarkers of T1D

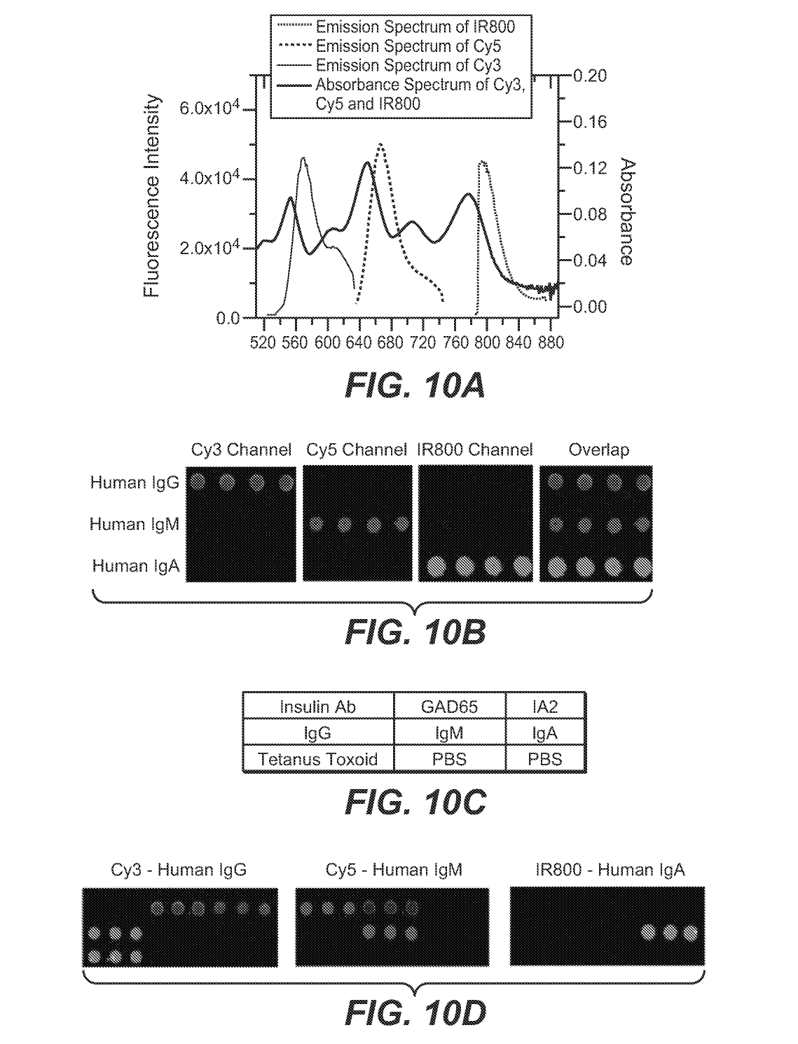

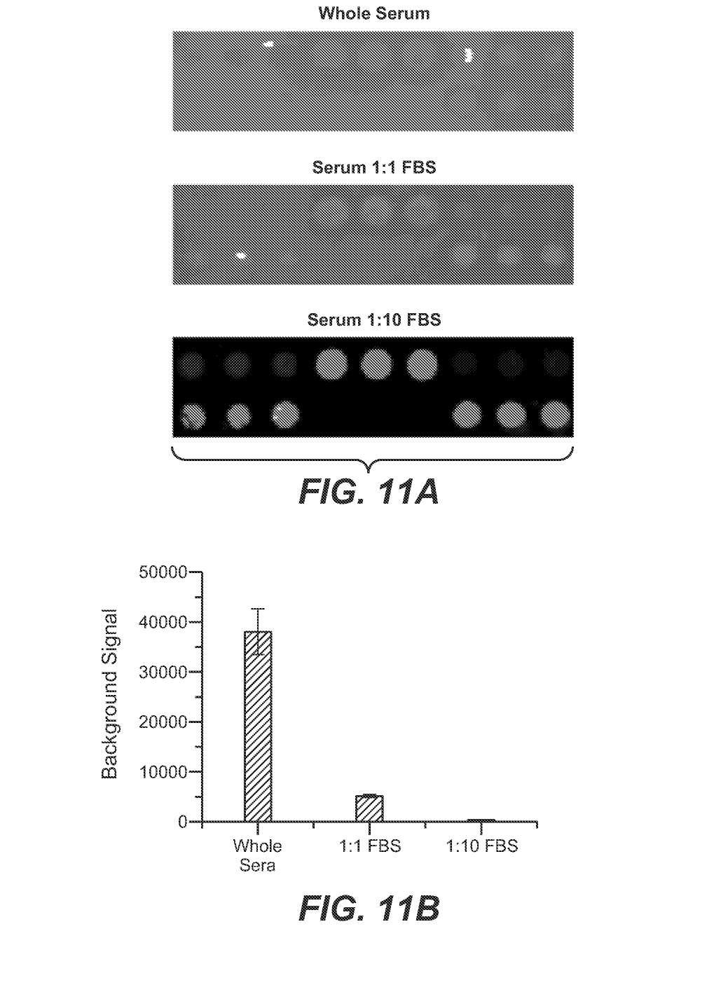

While modern RIAs favor IgG detection in their protein An immunoprecipitation step, they do not distinguish among immunoglobulin isotypes. IgM antibodies are produced early in an immune response and are later replaced by isotype switching to IgG antibodies. Taking advantage of our plasmonic chip's ability to enhance the fluorescence of multiple NIR dyes (e.g., Cy3, Cy5 and IRDye800 by ˜3, 50 and 100 fold respectively (FIG. 13) with non-overlapping emission spectra (FIG. 10), we achieved simultaneous detection of three subclasses of human autoantibodies (IgG/IgM/IgA) against each islet antigen (FIG. 10B). This was done by using Cy3 labeled anti-human IgG secondary antibody, Cy5 labeled anti-human IgM antibody and IRDye800 labeled anti-human IgA secondary antibody to probe the microarray spots of captured human IgG, IgM and IgA autoantibodies from a patient's serum. Note that we chose the dye with the lowest NIR-FE for reporting the antibody subtype in the highest abundance in human serum. This was the first time multi-color microarrays were developed on a plasmonic substrate. Using this multi-color detection scheme, we identified a new-onset diabetic patient with positive IgM autoantibodies against insulin and GAD65, in addition to positive IgG autoantibodies against GAD65 and IA2 (FIG. 10C). While further research is needed to elucidate if testing for IgM (or IgA) autoantibodies is clinically useful, this discovery exemplifies that the platform can be used as a new research tool enabling the testing of an enriched set of potential biomarkers compared to currently used panels.

### Example 7

### Materials and Methods (from Example 6)

Superfrost Plus glass slides were purchased from Fisher Scientific and rinsed with acetone, isopropanol (IPA) and methanol prior to use. Two-pad and Sixteen-pad Whatman FAST nitrocellulose slides, chloroauric acid trihydrate, hydroxylamine HCl, sodium borohydride, cysteamine, mercaptohexadecanoic acid, 1-ethyl-3-(3-dimethylaminopropyl) carbodiimide (EDC) and N-hydroxysuccinimide (NHS) were purchased from Sigma-Aldrich. Ammonium hydroxide (30% ammonia) and Hyclone fetal bovine serum were purchased from Fisher Chemicals. IR800cw-NHS ester was purchased from Licor Biosciences. 6-armed poly(ethylene glycol)-amine was purchased from SunBio. Fetal bovine serum was purchased from Invitrogen.

Recombinant human insulin was purchased from Lilly, GAD65 and IA2 (ICA512) antigens were purchased from Kronus and tetanus toxoid antigen was purchased from Santa Cruz Biotech. Goat anti-human IgG antibody and human IgG were purchased from Vector Lab.

Human Serum

Institutional Review Board approval for this study was obtained from Stanford University. Once parental consent and subject assent (when appropriate) were obtained, 2.5 mL of blood was drawn in a red-top venipuncture tube (silicon coated glass) and 2.5 mL in a lavender-top venipuncture tube (liquid K₂EDTA in glass). Samples were stored at 4° C. until processing. For processing, the 2.5 mL of whole blood collected in the lavender-top tube was divided into 125 μL aliquots in cryovials and stored at −80° C. The 2.5 mL collected in the red-top tube was centrifuged at 300 rpm for 15 min. The serum supernatant was then divided into 75 μL aliquots in cryovials and stored at −80° C. Samples were subsequently thawed and assessed in parallel.

Clinical Diagnosis and Monitoring of T2D Patients

All patients were monitored regularly for at least 6 months post diagnosis. Ten of the 12 patients categorized as T2D were transitioned completely off of insulin for over one month without developing ketosis or DKA and were able to maintain good blood glucose control with diet and exercise or diet, exercise and metformin. The other two patients had prolonged periods of insulin omission without ketosis and both of these patients tested negative for autoantibodies on both platforms. One patient tested positive for a novel mutation in the HNF4a gene (that has not been reported to be associated with MODY).

ELISA Assay Procedure

96-well microtiter plates (BD Falcon) were coated with 100 μg recombinant human insulin (Lilly) The antigen was incubated at 4° C. overnight followed by washing with PBST and then blocked overnight at 4° C. with 5% non-fat milk. 100 μL of 1:10 diluted serum for each subject was dispensed into the wells, and incubated overnight at 4° C. Samples were incubated with a 1:5000 dilution of rabbit-anti-human-IgG conjugated with horseradish peroxidase (Santa Cruz Biotechnology) for 2 hrs and then washed several times. Following plate washing, 50 μL of 3,3′,5,5′-tetramethylbenzidine (Sigma) was added to each well for 30 min, and then an equal volume of 2M H₂SO₄ (Sigma) was added to stop the substrate reaction. The optical density (450 nm) of each well was quantified using a plate reader (BioTek Synergy H1).

Preparation of Evaporated Gold Film on Glass Slides

Evaporated gold films were deposited via e-beam evaporation on an Innotec ES26C E-Beam Evaporator. The vacuum pressure of the system prior to deposition was 5e-7 Torr. The e-beam parameters were 10 kV at ˜0.1 A which resulted in a deposition rate of roughly 2 A/s.

Preparation of Plasmonic Gold Film on Glass Slides

Glass slides were immersed in 3 mM HAuCl₄ followed by adding ammonium hydroxide at 20 μL ammonium hydroxide per mL of HAuCl₄ solution with rapid shaking for one minute. The slide was washed twice with deionized (DI) water to remove unbound gold ions and immersed into 1 mM NaBH₄ to reduce gold clusters on the glass slide to gold nanoparticle seeds. After further washing twice with water, the slides were incubated in a solution of HAuCl₄ and hydroxylamine at a 1:1 ratio and shaken for 5 min, followed by a 10-min incubation to complete the growth step. After washing with water twice and drying, the slide was checked for plasmon resonance by using a Cary 300UV-Vis-NIR absorbance spectrometer after correcting for background absorbance from the glass substrate. Scanning electron micrographs were acquired on an FEI XL30 Sirion SEM with FEG source at 5 kV acceleration voltage.

Construction of Multilayer Surface Chemistry on Gold Film

Gold slides were immersed into 10 mM mercaptohexadecanoic acid in ethanol overnight at room temperature. After rinsing with ethanol and drying, the carboxylic group functionalized gold slide was immersed in a solution of 20 μM 6-arm poly (ethylene glycol)-amine (Mn ˜10,000 Da) and 20 mM each of 1-ethyl-3-(3-dimethylaminopropyl) carbodiimide and NHS in DMF. After rinsing the gold slide with dimethylformamide (DMF), ethanol and drying, the substrate was immersed in 10 mM succinic anhydride DMF solution with triethylamine at 1 μL per mL. This step transforms the free amine groups on the poly(ethylene glycol) chain into carboxylic groups. Following another washing step with DMF and ethanol, the slide was incubated in 20 mM each of 1-ethyl-3-(3-dimethylaminopropyl) carbodiimide and NHS in DMF to activate the carboxylic group.

Microarray Printing and Microarray Sandwich Assay Procedure

The NHS-activated gold slides above (or nitrocellulose slides, glass slides, NHS-activated evaporated gold slides) were loaded into a microarray printing robot (Bio-rad VersArray Chipwriter) where 3 μM antigens in PBS were printed using solid pins at 25° C. and 60% humidity, resulting in microarray feature diameters of ˜400 μm. The slides were dried in a desiccator and then blocked in phosphate buffered saline-Tween 20 (PBST) solution containing 5% fetal bovine serum. Human serum was diluted into FBS solution (1:10), and 20 μL of each solution was applied to each set of spots, and incubated (shaking) for 1 h at room temperature, followed by washing twice with PBST and once with PBS. The array was then incubated in 1 nM IR800 conjugated goat anti-human IgG in FBS for 20 min (shaking) at room temperature in the dark. Chips were washed twice with PBST and once with PBS, followed by immersion in DI water and subsequent drying with compressed air. The total processing time to test the human samples for autoantibodies using the plasmonic gold chips was less than 2 hours.

Fluorescence Measurement and Analysis

A Licor Odyssey scanner was used to scan the IRdye800-secondary antibody labeled microarrays on different substrates using the 800 nm channel with the gain set to 6.0 as defined by the system, and the resolution set to 42 μm. Microarray fluorescence images were analyzed by Genepix 6.1, and the spot features were automatically identified by the program. The fluorescence intensity of each spot was background corrected, and the average of mean pixel intensity values for features printed in duplicates. Note that the background signal was typically on the order of 10, while the spot signals were in the range of 10-100,000 for various autoantibody concentrations.

Calibration Curves for Insulin Ab, GAD Ab and IA2 Ab

For generating the calibration curves of each autoantibody, a serial standard samples with known amounts of autoantibody (Kronus) was applied to different microarrays on plasmonic chips, following the same procedure as described for human serum. Fluorescence signal vs. autoantibody concentration was plotted and was used for calibrating autoantibody concentrations in patient serum samples.

Statistical Analysis

We measured the mean fluorescence intensity (MFI) for each subject against insulin, GAD65 and IA2 (ICA512) islet antigens on the plasmonic gold platform, and directly compared these results to the known corresponding RIA data. Positive thresholds for the Au platform were determined by MFI measurements of control samples (obtained from the Kronus RIA kits) corresponding with the commercial Esoterix RIA positive threshold. We compared positive and negative MFI signals on our platform against RIA results to determine sensitivity and specificity for each diabetes autoantibody by receiver-operating characteristic (ROC) plots. Using subsequent clinical diagnosis determined from clinical progression and insulin needs at 6 months after disease presentation, we assessed each subject's diabetes autoantibody profile against their clinical diagnosis to compare the sensitivity and specificity of our platform against RIA as a diabetes diagnostic tool. We used a positive signal in any of the three autoantibodies as diagnostic of T1D. As the autoantibody profile included three MFI values in non-normal distribution, we conducted quadratic discriminate analysis of the subject data.

### Example 8

### Screening and Use of Antibody Detection

As described above, the present methods and devices provide a multiplexed assay in which a single human blood sample (or serum or urine) can be assessed for antibodies. These antibodies will have relevance to autoimmune aspects of TD 1 and related or similar diseases having autoimmune components. FIG. 3, similar to FIG. 8A, shows distinctions among different samples, where non-type 1 diabetes is distinguished. These assays are exemplified by the presence in the blood or serum of antibodies to GAD65, insulin and IA2. FIG. 10 shows a further detection of antibodies to an antigen such as insulin, GAD65 or IA2 by a detection reagent (anti-immunoglobin antibodies) specifically labeled to distinguish between IgG, IgM and IgA. This may be based on different Fc portions of different isotypes. Thus one could provide a multiplex detection having antigens, antibodies (in a sample), and detection reagents (applied to the samples) detecting specifically for isotypes of antibodies of interest. A chip could be constructed for detection as follows:

TABLE 3

| | Detection reagents for binding to sample | | | |
|---|---|---|---|---|
| Antigen on chip | antibodies to the cognate antigen | | | |
| GAD65 | Anti-IgG | Anti-IgM | Anti-IgA | Anti-IgE |
| GAD67 | “” | “” | “” | “” |
| IA512 | “” | “” | “” | “” |
| IA-2 | “” | “” | “” | “” |
| ZnT8 | “” | “” | “” | “” |
| Insulin | “” | “” | “” | “” |
| transglutaminase | “” | “” | “” | “” |

Various subsets of the matrix in Table 3, such as an assay using Anti-IgG and IgM against GAD 65, IA-2, and insulin. This is but one non-limiting example.

Such an array can be implemented in a plasmonic chip, as described above, or other electro-detection chips. Thus, there is no preparation required, and the results may be obtained within a day. Further, the assay is extremely sensitive, due to the increased fluorescence. In addition, embodiments can be created for subsets of the above markers where such sensitivity is not required. In addition, these platforms can use various fluidics including channels/microfluidics, automation (robotics), semi-automation (pipetting stations and software control) and can use solid substrates, such as suspension arrays. One may prepare, for example, a fluid assay that simply detects IgM antibodies to insulin, GAD 65, and IA-2, or various isotypes of these antibodies.

The present method for characterizing autoantibody isotypes can be implemented in assays providing one or more antigens of interest such as insulin, GAD65, and fragments of islet antigens from individuals with T1D. One, a 40-kDa fragment from the intracellular portion of a tyrosine phosphatase-like protein (PTPRN gene), now referred to as IA-2ic or ICA512ic. The antigens are arrayed in detection substrates, such as glass slides, silicon, microwells, nitrocellulose or PVDF membranes, and magnetic and other microbeads. While microdrops of protein delivered onto planar surfaces are the most familiar format, there are a number of more advanced architectures incorporating developments in microfluidics and nanotechnology. Particles in suspension can also be used as the basis of arrays, providing they are coded for identification [e.g. Luminex, Bio-Rad systems. or differential display, capture (e.g. antibody) arrays can be probed with fluorescently labelled proteins from two different cell states, in which cell lysates are directly conjugated with different fluorophores (e.g. Cy-3, Cy-5) and mixed, such that the color acts as a readout for changes in target abundance. Use of 2-color assays with direct labelling allows control for spot variability as well as comparison between two different samples. Fluorescent readout sensitivity can be amplified 10-100 fold by tyramide signal amplification (TSA). Planar waveguide technology enables ultrasensitive fluorescence detection, with the additional advantage of no intervening washing procedures. High sensitivity can also be achieved with suspension beads and particles, using phycoerythrin as label or the properties of semiconductor nanocrystals (Quantum dots). A number of novel alternative readouts have been developed, including adaptations of surface plasmon resonance (for real time kinetic measurements), mass spectrometry (for definitive protein identification), resonance light scattering and atomic force microscopy (for nanoarrays).

Isotyping of autoantibodies in a sample can include identification of antibody class (IgG, IgA, IgM or IgE) and subclasses IgG1, IgG2, IgG3 and IgG4. Isotyping may be carried out by secondary antibodies that detect specific Fc portion of individual antibody classes. Anti-Fc antibodies are described e.g. in “Method for the preparation of anti-receptor antibodies,” U.S. Pat. No. 4,683,295. Suitable antibodies are also commercially available, such as from Pierce Antibodies (unit of Thermo Scientific), listed at http(colon slash slash) www (dot) pierce-antibodies.com/targets/t/HumanSecondaryAntibodies.cfm.

Alternatively, one may examine a subject's blood directly, as described e.g. in US 20130078633, “Detection of Isotype Profiles as Signatures for Disease.” As disclosed there:

Methods that generally involve detecting isotypes comprise: the steps of obtaining a peripheral whole blood sample from a subject, isolating RNA from the peripheral whole blood sample, or fraction thereof (e.g., peripheral blood mononuclear cells), reverse transcribing the isolated RNA using target specific primers to generate immunoglobulin cDNA transcripts, amplifying the immunoglobulin VDJ to Ig constant regions using multiplex PCR techniques, sequencing the amplicons, and analyzing the sequence data. Data analysis includes the steps of extracting Ig constant region sequence for each isotype and comparing the total number of all Ig isotype sequences for a given sample.

The present methods can be used to evaluate individuals exhibiting diabetes symptoms, such as of increased thirst and urination, and/or high blood glucose level. Positive findings of insulin and/or GAD antibodies can confirm or rule out a diagnosis of T1D. Insulin can be administered if T1D is found. Asymptomatic individuals can be tested for these markers in order to assess a status of an individual for future development of T1D, or to determine an individual's suitability for a clinical trial. Further, detection of IgM may reveal antibodies that were missed by other methods that bias IgG (such as RIA testing).

The present methods can be used in screening for diabetes in the general or a select population. The methods can be used for predicting diabetes in the general population or a select population; diagnosing diabetes; or monitoring diabetes (by following the titers of the antibodies). The present methods can be used to base treatment decisions tailored to a likelihood of developing T1D.

The present methods can be directed towards a variety of isotype determinations. For example, in celiac disease, inflammatory bowel disease, arthritis, asymatic ATA+, etc., there are conditions where one may conduct an antibody isotype analysis according to the present method. For example, in the case of celiac disease, the subject will possess anti-transglutaminase antibodies (ATA). ATA as IgA antibodies are more frequently found in celiac disease (CD); however, ATA IgG antibodies are found in CD and at higher levels when an affected individual had the IgA-less phenotype. One could study this by distinguishing ATA IgG and IgA using the present methods.

### CONCLUSION

The above specific description is meant to exemplify and illustrate the invention and should not be seen as limiting the scope of the invention, which is defined by the literal and equivalent scope of the appended claims. Any patents or publications mentioned in this specification are intended to convey details of methods and materials useful in carrying out certain aspects of the invention which may not be explicitly set out but which would be understood by workers in the field. Such patents or publications are hereby incorporated by reference to the same extent as if each was specifically and individually incorporated by reference and contained herein, as needed for the purpose of describing and enabling the method or material referred to.

1. A biosensor for use in a spectroscopic detection system, comprising:
(a) a substrate;
(b) a discontinuous gold film applied to said substrate, said gold film having plasmonic nano-islands of gold grown on gold seeds; and
(c) an array of antigens disposed in discrete locations and coupled to the discontinuous gold film, whereby emission from a label bound to an analyte capture agent is enhanced by the discontinuous gold film, wherein said antigens are at least two of
(i) GAD65 (glutamic acid decarboxylase-65 kDa);
(ii) GAD67 (glutamic acid decarboxylase-67 kDa);
(iii) IA512 (islet cell autoantigen 512);
(iv) IA-2 (insulinoma antigen 2);
(v) ZnT8 (zinc transporter 8); and
(vi) human insulin or an immunologically active fragment thereof.

2. The biosensor of claim 1 wherein said antigens comprise purified recombinant proteins.

3. The biosensor of claim 2 wherein the purified recombinant antigens are at least three of, at least four of, at least five of, or at least six of, the antigens selected from antigens (i) through (vi).

4. The biosensor of claim 1 wherein the antigens are chemically linked to the plasmonic nanoislands by a branched polyethylene glycol.

5. The biosensor of claim 1 wherein the antigens further include an antigen that is reactive to antibodies raised by a vaccine.

6. The biosensor of claim 5 wherein said antigen is tetanus toxoid.

7. The biosensor of claim 1 further comprising channels on the biosensor for delivering reagents to said array of antigens.

8. The biosensor of claim 7 further comprising a separation zone for separating blood components and passing serum containing autoantibodies through the channels, whereby whole blood can be introduced into the biosensor.

9. The biosensor of claim 7 wherein said channels, in use, contain an anti-human IgG composition.

10. A method for determining an antibody specificity profile in a patient having a predisposition to insulin-dependent diabetes mellitus (IDDM), comprising:
(a) providing a protein array comprising at least three purified proteins selected from the group consisting of:
(i) GAD65 (glutamic acid decarboxylase-65 kDa);
(ii) GAD67 (glutamic acid decarboxylase-67 kDa);
(iii) IA512 (islet cell autoantigen 512);
(iv) IA-2 (insulinoma antigen 2);
(v) ZnT8 (zinc transporter 8); and
(vi) human insulin or an immunologically active fragment thereof,
said array coupled to a plasmonically active gold film in individual spots of arrayed proteins;
(b) contacting the array from step (a) with a patient sample comprising antibodies;
(c), identifying antigens among the arrayed proteins that bind to antibodies within the patient sample contacted in step (b) with a labeled antibody that binds to human antibodies and carries a fluorescent dye whose fluorescence is enhanced by the plasmonically active gold film.

11. The method of claim 10 further comprising reading fluorescence in a fluorescent reader that directly exposes the array to NIR and receives NIR reflected from individual spots.

12. The method of claim 10 further comprising the step of quantifying levels of antibody to said identified antigens.

13. The method of claim 10 wherein said at least three purified proteins are recombinant proteins.

14. A method for determining an antibody profile in a subject, comprising:
(a) providing a biosensor for use in a spectroscopic detection system, comprising:
a substrate;
a discontinuous gold film applied to said substrate, said gold film having plasmonic nano-islands of gold grown on gold seeds; and
an array of antigens disposed in discrete locations and coupled to the discontinuous gold film, whereby emission from a label bound to an analyte capture agent is enhanced by the discontinuous gold film, wherein said antigens are at least two of
(i) GAD65 (glutamic acid decarboxylase-65 kDa);
(ii) GAD67 (glutamic acid decarboxylase-67 kDa);
(iii) IA512 (islet cell autoantigen 512);
(iv) IA-2 (insulinoma antigen 2);
(v) ZnT8 (zinc transporter 8);
(vi) human insulin protein or an immunologically active fragment of said insulin protein; and
(vii) an antigen reactive to antibodies commonly found in humans;
(b) contacting said array of antigens with a subject sample containing antibodies;
(c) preparing complexes of antigens binding to antibodies in step (b);
(d) labeling complexes from step (c) with a florescent label enhanced by the plasmonically active gold film; and
(e) measuring fluorescence level of said complexes as labeled in step (d) and comparing florescence levels between at least two antigens selected from (i) through (vii).

15. The method of claim 14 further comprising the step of testing a subject for predisposition to insulin-dependent diabetes mellitus (IDDM).

16. The method of claim 15 wherein having testing comprises HLA-DR3 and DR4 antigen testing.

17. The method of claim 14 further comprising reading fluorescence in a fluorescent reader that exposes the array to near-infra red (“NIR”) and receives and measures NIR reflected from said discrete locations.

18. The method of claim 14 wherein said array comprises at least three antigens.

19. The method of claim 18 wherein said antigens are IA512, GAD65, and insulin.

20. The method of claim 19 further comprising the step of comparing levels of autoantibodies among IA512, GAD65, and insulin.

21. The method of claim 14 wherein the complexes include antibodies that are isotype specific.

22. The method of claim 21 comprising separately labeling human IgG and at least one of human IgM, human IgE, and human IgA.

23. The method of claim 14 wherein one determines one or more of
IgG and IgM subclasses of subject's antibody to GAD65;
IgG and IgM subclasses of subject's antibody to insulin;
IgG and IgM subclasses of subject's antibody to IA-2; and
IgG and IgA subclasses of subject's antibody to transglutaminase.

24. A biosensor kit for use in a spectroscopic detection system, comprising:
(a) a substrate having a discontinuous gold film applied to said substrate, said gold film having plasmonic nano-islands of gold grown on gold seeds;
(b) an array of antigens disposed in discrete locations and coupled to the discontinuous gold film, whereby emission from a label bound to an analyte capture agent is enhanced by the discontinuous gold film, wherein said antigens are at least three of
(i) GAD65 (glutamic acid decarboxylase-65 kDa);
(ii) GAD67 (glutamic acid decarboxylase-67 kDa);
(iii) IA512 (islet cell autoantigen 512);
(iv) IA-2 (insulinoma antigen 2);
(v) ZnT8 (zinc transporter 8); and
(vi) human insulin or an immunologically active fragment thereof; and
(c) a composition of detecting antibodies for detecting sample antibodies in a sample, said detecting antibodies being of classes of at least two of human IgG, IgM, and, IgA subclasses, and said detecting antibodies comprising an NIR label having different emission characteristics for different detecting antibodies binding to different classes of sample antibodies.

25. The kit of claim 24 wherein said NIR label includes IRDye 800 (emission max 806), Cy5 (emission max 670), and Cy3 (emission max 570).

26. The kit of claim 24 further comprising an NIR-field emission detection device.

27. The kit of claim 24 wherein said array comprises spots on an inert substrate and said spots include both antigens selected from antigens listed in (i) through (vi) and control spots.

28. The kit of claim 24 further comprising detection reagents specific to human Fc portions of at least IgG and IgM.

29. The kit of claim 24 wherein said antigens are linked to said substrate by PEG.
